(19) United States
(12) Patent Application Publication
(10) Pub. No.: US 2020/0362314 A1
(43) Pub. Date: Nov. 19, 2020

(54) METHODS TO PRODUCE ADULT-LIKE CARDIOMYOCYTES DERIVED FROM INDUCED PLURIPOTENT STEM CELLS FOR DRUG DISCOVERY

(71) Applicant: THE REGENTS OF THE UNIVERSITY OF COLORADO, A BODY CORPORATE, Denver, CO (US)

(72) Inventors: Kunhua Song, Centennial, CO (US); Walter Knight, Denver, CO (US); Keith Koch, Erie, CO (US); Timothy McKinsey, Broomfield, CO (US)

(21) Appl. No.: 16/875,829

(22) Filed: May 15, 2020

Related U.S. Application Data
(60) Provisional application No. 62/849,823, filed on May 17, 2019.

(51) Int. Cl. C12N 5/074

(52) U.S. Cl. CPC C12N 5/0696; C12N 2500/32; C12N 2500/35; C12N 2500/34

(57) ABSTRACT

Human induced pluripotent stem cell derived cardiomyocytes (hiPSC-CMs) are a powerful tool for studying cardiovascular disease and drug screening. However, most current methods of cell culture result in cells resembling fetal myocardium, which is potentially problematic as most forms of cardiovascular disease occur in the adult heart. Disclosed systems and methods include culturing cells in fatty-acid based medium and on patterned growth to produce hiPSC-CMs which mimic adult cardiomyocytes and display a similar hypertrophic response as is observed in cardiovascular disease.

### CROSS REFERENCE TO RELATED APPLICATIONS

The present application claims the benefit of priority to U.S. Provisional Patent Application No. 62/849,823, entitled “Methods to produce adult-like cardiomyocytes derived from induced pluripotent stem cells for drug discovery,” filed May 17, 2019, the entirety of which is hereby incorporated by reference herein for all purposes.

### STATEMENT REGARDING FEDERALLY SPONSORED RESEARCH OR DEVELOPMENT

This invention was made with government support under grant numbers 5T32H L007822-20 and R01HL133230 awarded by the National Institutes of Health. The Government has certain rights in the invention.

### FIELD

The disclosed processes, methods, and systems are directed to cardiomyocytes derived from induced pluripotent stem cells, and more specifically, to maturation of such cardiomyocytes.

### BACKGROUND

Cardiomyocytes derived from human induced pluripotent stem cells (hiPSCs-CMs) represent a powerful tool to study cardiovascular physiology and disease, offering a number of unique benefits. Significant differences exist between hearts of humans and the small animals most often used for modeling cardiovascular disease, including beating rate, cell size, multinucleation frequency, and myosin heavy chain expression. Therefore, the capability to study cardiovascular disease in a human cell system is important.

hiPSC-CMs offer a number of benefits, including scalability, the ability to generate patient-specific cells, and to quickly create knockout and transgenic lines via CRISPR/Cas9-mediated gene editing. In spite of these benefits, hiPSC-CMs possess a significant drawback: they are physically and functionally immature, and typically resemble fetal or neonatal cardiomyocytes in terms of cell size and morphology, gene expression, myofibril contractility, and metabolic activity. While certain forms of cardiovascular disease, such as congenital heart defects and cardiomyopathies caused by homozygous or compound heterozygous mutations, do primarily affect infants, most forms of pathological cardiac remodeling are diseases that afflict the elderly. Therefore, the functional immaturity of hiPSC-CMs represents a potential barrier to modeling and investigating many forms of cardiovascular disease.

### SUMMARY

Disclosed herein are compositions, cells, methods, and systems for improving maturity of human induced pluripotent stem cell derived cardiomyocytes as a platform for modeling cardiovascular disease.

Disclosed herein are surprising data showing that combination of growth of hiPSC-CMs in fatty acid media on a novel patterned surface induces CM maturity that is not possible if the cells are grown in either condition alone. Moreover, the mature induced CMs display characteristics that are unexpected from the existing protocols (for example, protocols that induce CMs using media containing glucose or fatty acids, without a patterned growth surface, or those using a patterned growth surface without a fatty acid media). Finally, the disclosed cells, devices, methods, and systems are useful in generating data that closely mimics explant tissue.

The cells, devices, methods, and systems disclosed herein are useful in producing cardiomyocytes that are more mature than those obtained from existing methods, and are cells with unexpected characteristics compared to cells grown either in fatty acid-containing media alone, or cells grown on patterned surfaces alone. Together, the disclosed methods and systems are useful in inducing a number of independent aspects of cardiomyocyte maturity that result in cells that more accurately mimic mature cardiomyocyte cells in-vivo. Thus, the disclosed cells, devices, methods, and systems are useful in disease modeling that more accurately reflects the in-vivo disease condition.

The disclosed cells, devices, methods, and systems induce maturity of hiPSC-CM that may be reflected in one or more of circularity, sarcomere elongation, and sarcomere organization, that is greater than that of sarcomeres in cells produced by other methods, for example growth in glucose (GLUC) or fatty acid (MM) alone. In some embodiments, the highest average sarcomere length is about 1.9-2.0 μm, which is similar to that of human adults sarcomeres. In many embodiments, the disclosed sarcomeres are more organized than those in cells produced from other methods, and may be observed by electron microscopy. In addition, myofibrils isolated from the disclosed cells cultured with the disclosed methods and systems are capable of generating a force that is similar to those from myofibrils isolated from adult human tissue. In many cases, these characteristics are indicative of advanced myofibril maturity compared to existing methods.

The disclosed hiPSC-CMs express genes that are indicative of enhanced maturity, compared with cells produced from other methods. In some embodiments, the disclosed cells, methods and systems result in downregulation of genes involved in cell division and the cell cycle. Here again, as adult cardiomyocytes are nondividing, this gene expression pattern indicates that the disclosed methods and systems result in hiPSC-CMhiPSC-CMs that are more mature than cells produced from existing methods. In many embodiments, the disclosed hiPSC-CM show the highest expression of fatty acid oxidizing genes, increased levels of fatty acids, increased CPT activity, and a cardiolipin profile that is most similar to cells from human adults and higher than those found in cells produced from other methods, such as growth in glucose(GLUC).

Existing methods of maturing cardiomyocytes result in activation of hypertrophic signalling, in contrast to the presently disclosed methods. Specifically, prolonged growth of hiPSC-CMs under standard conditions (that is no growth on patterned surfaces, and growth in glucose-based medium) is sufficient to activate hypertrophic signaling. As activation of these pathways is a component of pathological cardiac remodeling, this represents a significant barrier to using existing populations of induced cardiomyocytes for study of cardiovascular disease and/or discovery of therapeutic compounds for treating same. By contrast, the disclosed methods and systems suppress this signaling, sensitizing the disclosed hiPSC-CM cells to either hypertrophic stimuli and cardiomyopathy-causing mutations, as well as to compounds that reverse these effects. Specifically, hypertrophic agonists (for example phenylephrine ‘PE’) was shown to increases the size of the presently disclosed cells and B-type natriuretic peptide ‘BNP’ expression (hypertrophy), and the antihypertrophic drug JQ-1 was shown to reverse this effect.

Contractile function of the presently disclosed hiPSC-CM cells was also markedly different upon treatment with phenylephrine. Specifically, PE treatment of the disclosed hiPSC-CMs shortened myofibril relaxation time and increased relaxation rate, which mimic changes observed in explanted heart tissue from adults with hypertrophic cardiomyopathy.

Human induced pluripotent stem cell derived cardiomyocytes (hiPSC-CMs) represent a valuable tool for characterizing cardiac physiology and pathology, and serve as the main human cell system available to cardiovascular biologists. However, hiPSC-CMs are functionally immature, closely resembling neonatal cells in terms of gene and protein expression, metabolic behavior, and contractile function. As cardiomyopathies are primarily diseases of the elderly, the immaturity of hiPSC-CMs presents a significant barrier to accurately modeling human cardiac disease.

To address this concern, disclosed methods and systems improve hiPSC-CM maturity. Disclosed systems and methods may include a fatty acid-based culture medium designed to mimic adult myocyte energy consumption together with patterned growth surfaces designed to promote adult myocyte cellular organization. In combination, these methods promote cellular elongation, anisotropy and sarcomere organization, and reduce the expression of hypertrophic/neonatal markers compared to cells cultured in standard glucose medium.

In some embodiments, RNA-seq and metabolic screening of the presently disclosed cells demonstrates increased expression of cardiomyocyte maturation markers and a metabolic shift from glycolysis towards fatty acid oxidation. To evaluate functional improvements in the presently disclosed mature hiPSC-CMs, contractile and relaxation mechanics were measured in isolated myofibrils. The presently disclosed methods result in significantly increased maximum myofibril tension generation, to a similar level as myofibrils isolated from adult human heart. To investigate the capability of the disclosed system to study pathological cardiac remodeling, the response of the disclosed cells to the hypertrophic agonist phenylephrine (PE) was tested. PE treatment induced a significant increase in cell area in hiPSC-CMs grown under the presently disclosed methods and conditions, to a similar degree as reported in adult mouse cardiomyocytes. PE treatment also induced changes in myofibril relaxation in the disclosed cells, similar to reports in human hypertrophic cardiomyopathy. Taken together, compared to existing hiPSC-CM culture protocols, the disclosed systems, methods, and cells are able to optimize cell maturity, organization, and hypertrophic response, and therefore may represent a powerful system and method for the study of human cardiovascular disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an illustration depicting deriving cardiomyocytes from human donor fibroblasts. Fibroblasts from donors or patients are reprogrammed into induced pluripotent stem cells (iPSCs) using Yamanaka factors. By modulation of GSKβ and Wnt signaling, these iPSCs can then be converted into contracting cardiomyocytes, but these resemble neonatal cells.

FIG. 2 is a set of images and graphs showing images of human stem cell derived cardiac myocytes (Panel A), and mouse cardiocytes in vitro (Panel B) and in vivo (Panel C), showing lack of sarcomeric alignment and triangular shape in hiPSC-CMs. (Panel D), Maximum force in myofibrils from hiPSC-CMs is much lower than in human cardiac tissue (Mean +/−SEM, t-test). (Panel E) micrograph showing myosin heavy chain expression in tissue and hiPSC-CMs. Specifically, the hiPSC-CMs express predominantly beta-myosin heavy chain (β-MHC), as does human adult cardiac tissue.

FIG. 3 is an illustration and an image (Panel A) Basic (or GLUC) and improved iPSC-CM culture methods. Identical induction and selection protocols are used for each experiment; after selection, the ‘improved’ protocol consists of culturing cells in media containing fatty acid (or MM). ‘Improved+pattern’ protocol consists of replating cells on grooved surfaces and culturing in improved media (or MMPAT). (Panel B) Patterned surface for iPSC-CMs.

FIG. 4 is a set of images and graphs showing (Panel A) hiPSC-CMs cultured under basic (upper) or improved, patterned (lower) conditions. Improved media and patterned surfaces improve cell alignment, sarcomere organization, and ventricular myosin light chain (MLC-2v) expression. (Panel B) Quantification of circularity from hiPSC-CMs grown under each condition. Lower circularity indicates rectangular morphology. (Panel C) Additional images of hiPSC-CMs grown under optimal conditions, showing cell alignment and expression of myosin binding protein C3 and ryanodine receptor, which are expressed in mature cardiomyocytes. Data presented as mean +/−SEM.

FIG. 5 is a set of images and graphs showing (Panel A) Myofibril isolation rig. (Panel B) Myofibril isolated from hiPSC-CM, mounted between stretcher and force probe. (Panel C) Representative trace from myofibril activation/reactivation/relaxation. (D) Activation traces from myofibrils from hiPSC-CMs cultured under each condition. (Panels E-H) Maximum tension, resting tension, reactivation kinetics, and slow phase relaxation kinetics from hiPSC-CM-derived myofibrils cultured under each condition (Basic or GLUC; improved or MM; and improved+pattern or MMPAT). Data derived from 3-7 individual experiments per condition. Data presented as mean +/−SEM, significance testing performed by one way ANOVA followed by Dunnett's multiple comparison test.

FIG. 6 is a set of graphs showing (Panel A) Representative activation traces from myofibrils derived from a human donor and a human HCM heart. (Panels B-H) Mechanical data from human myofibrils. Maximum and resting tension, activation and reactivation constants, fast and slow phase relaxation constants, and linear phase relaxation time are shown. Defects in relaxation may be indicative of diastolic dysfunction. Data derived from 5-8 individual hearts per condition. Data presented as mean +/−SEM, significance testing performed by t-test.

FIG. 7 is a summary illustration showing that this combination of culturing methods improve hiPSC-CM morphology and maturity.

FIG. 8 shows images and graphs depicting morphological improvements with maturation methods in hiPSC-CMs. Panel A shows representative images of staining for various sarcomeric proteins in hiPSC-CMs cultured in each condition, including cTNI: Cardiac troponin I; α-act: α-actinin; MLC-2V: Myosin light chain, ventricular isoform; and MYBPC3: myosin binding protein C3. White arrows indicate areas lacking sarcomeres. Panel B is a graph depicting average cell areas in hiPSC-CMs cultured under each condition. Data pooled from 3 independent experiments, 100-250 cells assessed per condition per experiment. Panel C is a graph depicting cellular circularity (defined by circularity=(4*π*area/perimeter2) in hiPSC-CMs cultured in each condition. Data from 4 independent experiments, 100-250 cells assessed per condition per experiment. A lower circularity is indicative of more elongated objects. **, ****, P<0.01, 0.0001, Kruskal-Wallis test with Dunn's multiple comparison test (Panel B), or one way ANOVA with Tukey's multiple comparison test (Panel C).

FIG. 9 shows images and graphs depicting sarcomere morphology improved with maturity methods. Panels A-B show transmission election microscopy images of hiPSC-CMs cultured under each condition. Red boxes in (Panel A) are enlarged in (Panel B). Red arrows indicate I-bands, blue arrows indicate H-zones. Panel C is a graph depicting sarcomere length in hiPSC-CMs cultured in each condition. Sarcomeres measured between Z-lines, from 150-200 cells per condition per experiment, from 5 independent experiments. *P<0.05, one way ANOVA with Tukey's multiple comparison test.

FIG. 10 shows images and graphs depicting myofibril mechanics in hiPSC-CMs and human heart tissue. Panel A shows representative images of myofibrils isolated from each group and cells and from human donor hearts, mounted between stretcher (left) and force probe (right). Blue arrows indicates striations. Panel B shows a heatmap of hiPSC-CM myofibril mechanics properties. Unsupervised hierarchical clustering by complete linkage performed on columns, indicating that MPAT cells cluster most closely with human adult donor myofibrils. Panel C is a graph depicting average myofibril maximum tension generation, normalized to myofibril area. *,***, **** P<0.05, 0.001, 0.0001, one way ANOVA with Tukey's multiple comparison test on log2-transformed data. 24-31 myofibrils were isolated from hiPSC-CMs, pooled from 4 independent cell inductions, while 6 myofibrils were isolated from the donor heart. For a complete set of myofibril mechanics data, see Table 1. Panel D shows an image of western blots on hiPSC-CMs lysates for myosin light chain ventricular isoform (MLC-2V), myosin light chain atrial isoform (MLC-2A), myosin binding protein C3 (MYBPC3), α-actinin, cardiac (C-TNI) and slow skeletal Troponin I (ss-TNI), myosin II, and alpha tubulin as loading control. 4 μg protein loaded per well.

FIG. 11 shows graphs showing how disclosed maturation methods induce broad changes in hiPSC-CM metabolitic behavior. Panel A is a graph depicting qPCR for selected genes involved in mitochondrial fatty acid uptake and metabolism and cardiolipin remodeling, normalized to 18S rRNA and shown as fold change relative to GLUC. Panels B-C show graphs depicting selected results from metabolic screening in hiPSC-CMS, parsed by metabolite type. Screening was performed on cells cultured in each maturation condition and expressed as fold change relative to GLUC hiPSC-CMs. Heatmaps of all assayed metabolites and of differentially expressed metabolites can be seen in FIG. 18. Panel D is a graph depicting CPT1 and CPT2 activity in hiPSCs and hiPSC-CMs cultured under each condition. Panel E is a graph depicting quantification of peak areas of cardiolipin side chain percentages in hiPSCs and hiPSC-CMs cultured under each condition. *, **, ***, **** p<0.05. 0.01, 0.001, 0.0001, one way ANOVA followed by Tukey's multiple comparison test. For panels A-C, tests performed on log2-transformed data. Cells or cDNA prepared from four independent inductions were used for panels A-C, while cells from three independent inductions were used for panels D and E.

FIG. 12 shows images and graphs depicting results indicating that culture in maturation medium allows induction of a hypertrophic response in hiPSC-CMs. Panels A-B show an image and graph, respectively, depicting hiPSC-CMs cultured under indication conditions and treated with vehicle, 10 μM phenylephrine (PE) or 10 μM PE and 1 μM JQ-1 (a BET Bromodomain inhibitor) for 48 hours, then fixed and stained for α-actinin (green) and Hoescht 3342 (blue). Representative images are shown in panel A. Cell area of hiPSC-CMs were quantified in panel B. Cell areas measured on 616-789 cardiomyocytes pooled from four independent inductions. Panel C is a graph depicting qPCR for B-type natriuretic peptide, normalized to 18S rRNA and shown as fold change relative to GLUC, from cDNA prepared from four independent inductions. Panels D-F depict data showing characterization of cardiac hypertrophy in Danon hiPSC-CMs under various culture conditions, and their comparison to the hypertrophy observed in human patients. In Panel D, hypertrophy (as assessed by left ventricular posterior wall thickness, LVPW) in the hearts of human patients is shown. Panel E shows representative images of hiPSC-CMs cultured under indicated conditions, fixed, and stained with CellMask Orange(red) and Hoescht 3342 (blue). Panel F shows quantification of cell area of hiPSC-CMs derived from either a control line, or from patients with Danon disease (MD-111, MD-186), and cultured under the indicated conditions. Cell areas measured on 356-1056 cardiomyocytes pooled from two independent inductions. *, **, **** p<0.05, 0.01, 0.0001, Kruskal-Wallis test followed by Dunn's multiple comparison test (B,D) or one way ANOVA followed by Tukey's multiple comparison test on log2 transformed data (C).

FIG. 13 is graphs showing results that indicate that phenylephrine treatment induces changes in myofibril relaxation which are similar to those observed in human hypertrophic cardiomyopathy. Panel A shows graphs depicting myofibril mechanics data from human donor and idiopathic hypertrophic cardiomyopathy (HCM) hearts. Myofibrils were isolated from 4 donor and 4 HCM hearts, with at least 6 myofibrils isolated per heart. Panel B shows graphs depicting MPAT hiPSC-CMs treated with either vehicle or 10 μM PE for 48 hours Myofibril mechanics data from MPAT hiPSC-CMs treated with either vehicle or 10 μM PE for 48 hours. 44-45 myofibrils isolated per condition, pooled from 3 independent inductions. Complete tables of myofibril data from these experiments are available in Tables 4 and 5. *, **, **** p<0.05, 0.01, 0.0001, Student's t test on log2-transformed data.

FIG. 14 shows images depicting attachment of hiPSC-CMs to patterned surfaces. Brightfield images of cells at various time points after plating. Cells can be seen to begin to attach and align to grooves within 3 hours of plating, with the majority of cells having aligned within 24 hours, and nearly all cells after 48 hours. After several days of plating, cells plated at high density form a sheet with coordinated contraction along the direction of micropatterning.

FIG. 15 shows graphs depicting results that indicate fatty acid medium and patterning improves hiPSC-CM maturity. Panel A is a graph comparing cell area distribution of hiPSC-CMs cultured under each condition, and adult mouse ventricular cardiomyocytes. Data pooled from 507-706 cardiomyocytes per condition from 4-5 independent experiments. Panel B is a graph showing percentage of cells positive for ventricular myosin light chain (MLC-2V) in each culture condition. Data from 3 independent experiments, 75-325 cells assessed per condition per experiment. Panel C is a graph depicting mean α-actinin staining fluorescence intensity per cell, normalized to glucose-cultured hiPSC-CMs. Data from 5 independent experiments, 100-150 cells assessed per condition per experiment. *P<0.05, one way ANOVA with Tukey's multiple comparison test. N.S., nonsignificant. Data are mean+SEM.

FIG. 16 is an image depicting hiPSC-CM protein expression and myofibril mechanics. Blue silver staining of myosin heavy chains (MYH6 and 7, indicated by arrows) in hiPSC-CMs, as well as from a donor human heart lysate (as a positive control for MYH7) and a 12 week old mouse heart (positive control for MYH6). In a single MM hiPSC-CM sample, significant MYH6 could be detected (indicated by thick arrow); all other samples appear to express exclusively MYH7.

FIG. 17 shows images of RNA-seq on hiPSC-CMs cultured under each condition. Panel A shows an image of a heatmap of all genes with >1.5 fold change (increase or decrease in expression) between GLUC and MPAT cells. Panel B shows a diagram of overlap in genes displaying >1.5× increase (left panel) or decrease (right panel) in expression in indicated comparison. Many of the same genes are upregulated or downregulated comparing GLUC versus MM and GLUC versus MPAT, whereas other comparisons have relatively little overlap. Panel C shows an image of a heatmap of selected genes involved in fatty acid oxidation. Unsupervised hierarchical clustering performed on both rows and columns, by centroid linkage.

FIG. 18 shows images depicting results that indicate maturation methods induce broad changes in hiPSC-CM metabolites. Panel A is an image of a heatmap of all polar metabolites detected in screening, showing individual experimental replicates from each separate cardiomyocyte induction. Panel B is an image of a heatmap of significantly differentially expressed metabolites, as assessed by one way ANOVA and Tukey's post test on log2 transformed data. Unsupervised hierarchical clustering performed on both rows and columns, by centroid linkage.

FIG. 19 shows images of results indicating maturation methods induce changes in cardiolipin side chain content in hiPSC-CMs. Representative cardiolipin spectra from hiPSCs and hiPSC-CMs cultured under each condition. Peaks are labeled by total MW range of cardiolipins contained within peak.

FIG. 20 shows images and graphs depicting hypertrophic response in hiPSC-CMs and adult mouse ventricular cardiomyocytes (AMVCMs). Panels A-B show representative images of BNP staining and quantification of BNP and pro-NT-BNP-positive hiPSC-CMs cultured and treated as described in FIGS. 12A-B. Panels C-D show representative brightfield images and cell area of AMVCMs under indicated conditions. AMVCMs were isolated from 8-12 week old adult mice then treated with vehicle, 10 μM phenylephrine (PE) or 10 μM PE and 1 μM JQ-1 for 72 hours, then fixed. **** p<0.0001, Kruskal-Wallis test followed by Dunn's multiple comparison test. Cell areas measured on 664-706 cardiomyocytes pooled from four separate cardiomyocyte isolations.

FIG. 21 shows graphs depicting myofibril mechanics data on AMVCMs. Myofibril mechanics data from AMVCMs isolated from 8-12 week old adult mice and treated with vehicle or 10 μM PE for 72 hours before myofibril isolation. Note that rodent myofibrils display profoundly faster kinetics of both activation and relaxation at baseline compared to myofibrils from human hiPSC-CMs and cardiac tissue. *, p<0.05, Student t test. 21-26 myofibrils were isolated from three separate cardiomyocyte isolations. A complete table of myofibril mechanics data on AMVCMs can be seen in Table 6.

### DETAILED DESCRIPTION

Disclosed herein are methods and systems for producing structurally and functionally mature human induced pluripotent stem cell derived cardiomyocytes (hiPSC-CMs). hiPSC-CMs are a powerful tool for studying cardiovascular physiology and disease and drug screening. However, most current methods of cell culture result in immature cells resembling fetal myocardium, which is a barrier to modeling adult-onset cardiovascular disease. Disclosed herein are compositions, cells, methods and systems useful in inducing hiPSC-CMs towards maturity in vitro, for example, producing hiPSC-CMs which significantly resemble adult human cardiomyocytes in terms of metabolic and contractile function.

Disclosed systems and methods may include a combination of lactate selection, maturity-inducing culture medium which mimics the environment of the adult heart, and patterned growth surfaces to produce hiPSC-CMs which mimic adult cardiomyocytes. The maturity-inducing culture medium may be a fatty-acid based medium in which fatty acids, rather than glucose, represent the primary energy source. The patterned surfaces may be designed to promote cellular elongation and other structural and functional features of adult human cardiomyocytes.

The presently disclosed systems, methods, and compositions enhance maturation of the hiPSC-CMs by culturing the cells in a medium containing fatty acids as a significant energy source, combined with plating the cells on micropatterned growth surfaces. The presently disclosed cells demonstrate elongation, enhanced sarcomeric maturity, metabolic gene expression, mitochondrial fatty acid uptake, and cardiolipin maturation. Moreover, the mechanical behavior of myofibrils isolated from these cells closely resembles those from adult human ventricular tissue, particularly in terms of force generation. These mature hiPSC-CMs also respond well to hypertrophic stimulation, producing an adult-like hypertrophic response, and demonstrating alterations in myofibrils relaxation similar to human HCM tissue.

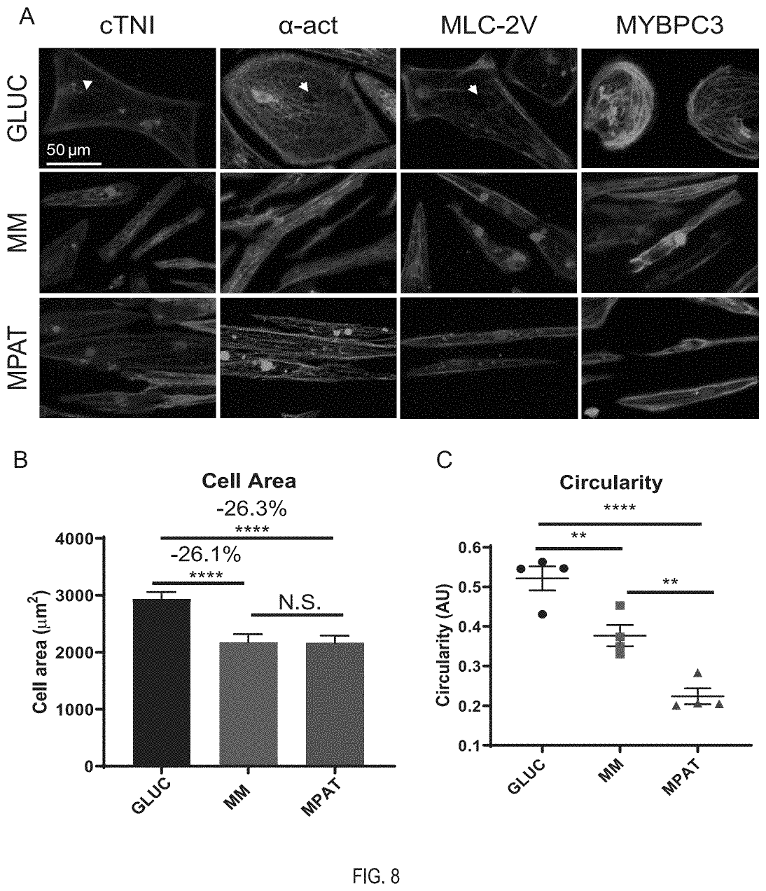

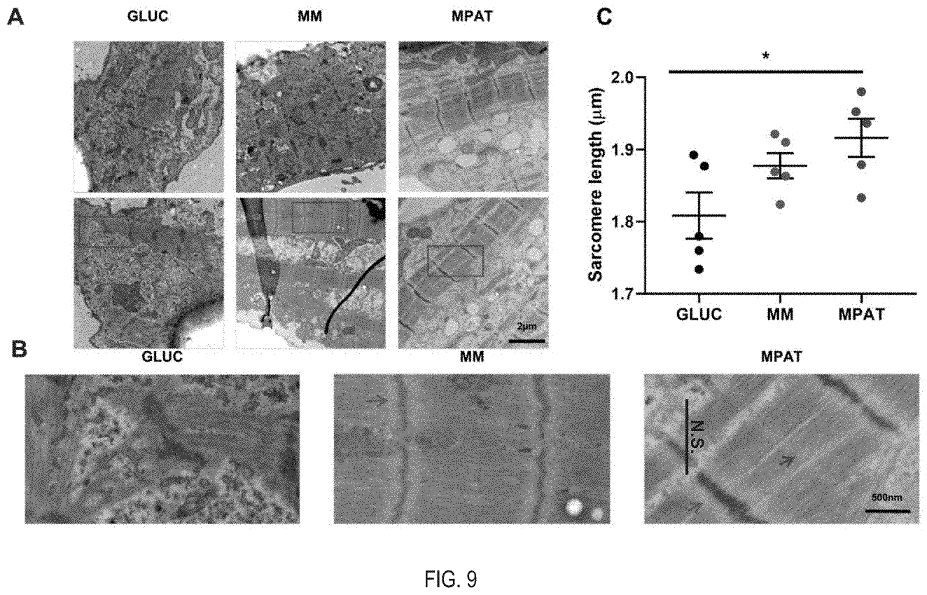

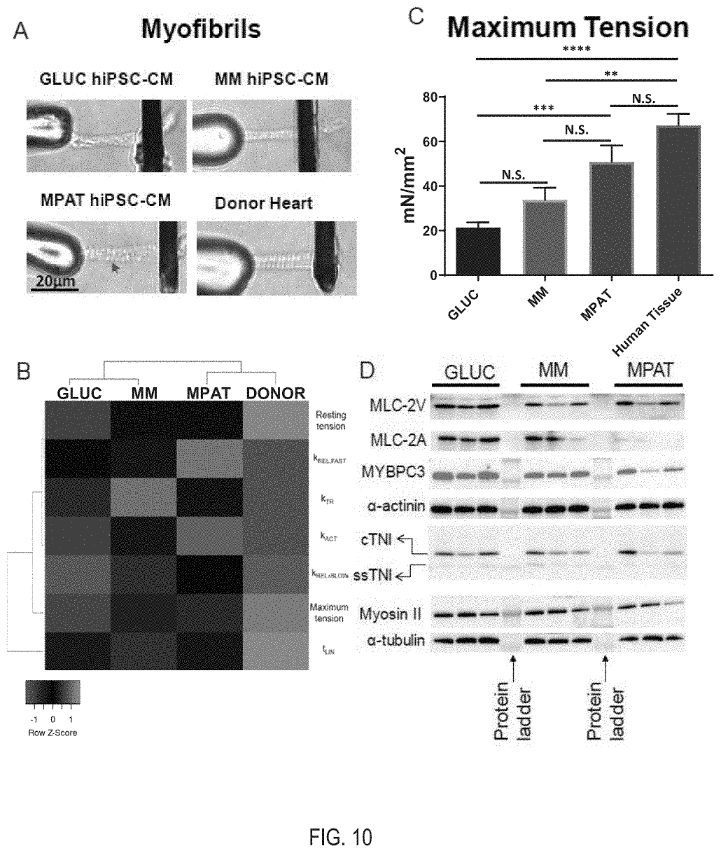

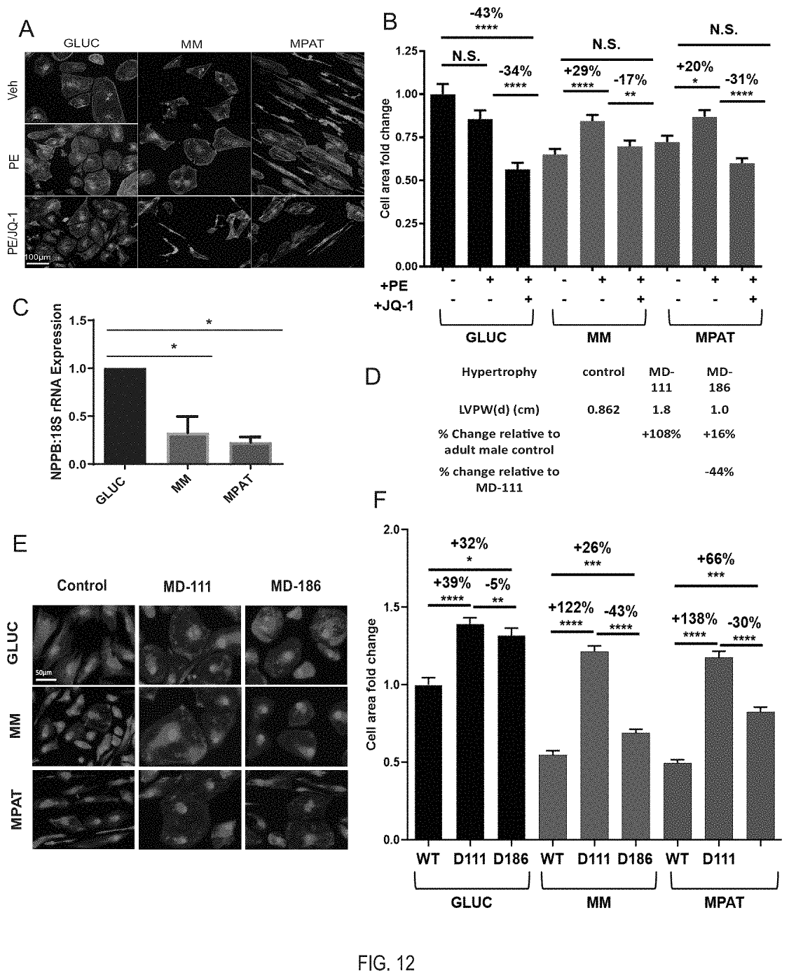

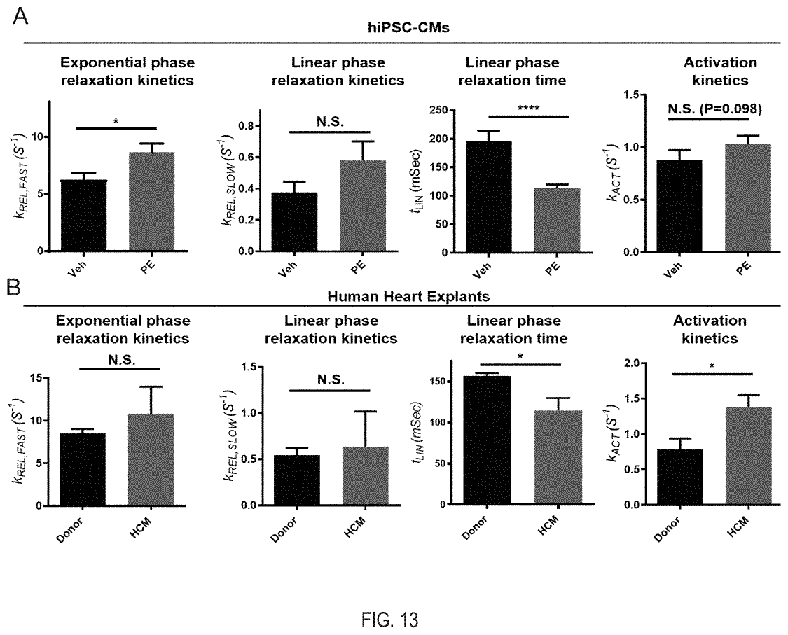

In the past several years, improving hiPSC-CM maturity has been a topic of acute interest, with dozens of studies reporting a plethora of methods of achieving this aim, including prolonged culture, electrical pacing, treatment with T3 hormone, and engineered heart tissue setups. The presently disclosed combinatorial approach may provide significant benefit over the existing approaches, in part, due to the simplicity of the present compositions, methods, and systems. For example, fatty acid medium can be prepared in a large scale at low cost, while patterned surfaces require only a few simple tools to produce. The presently described culturing setup can therefore likely be introduced into new research environments quite quickly and with low cost. The presently disclosed combinatorial methods also have the benefit of inducing a more mature cardiac phenotype in the resulting hiPSC-CMs across a variety of parameters, from cell morphology (FIGS. 8, 9) to contractility (FIGS. 10, 13), and hypertrophic response (FIGS. 12, 13, 20). Therefore, the presently disclosed combinatorial method, systems, and the resulting cells are useful in a diverse set of future applications.

Without wishing to be limited by theory, the presently disclosed systems, methods, and compositions result in maturation steps that appear to exert their benefits on different aspects of cardiac maturity. For example, many of the improvements observed in myofibril mechanics occurred in hiPSC-CMs produced by combining the maturation medium with the patterned surface, as compared with those produced with the maturation medium alone. This shows that the presently described combinatorial approaches may be beneficial in producing mature hiPSC-CMs that more closely resemble adult human cardiomyocytes. Compositions, methods, and systems disclosed herein may be useful for various applications, such as, for example, screening for new cardiac drugs or analyzing the effect of genetic or pharmacological perturbations in mature human cardiac cells. The presently disclosed compositions, methods, and systems may also be combined with other methods, such as cell pacing, growth hormone treatment, or use of altered ECM composition or stiffness to promote further maturation.

Cell pacing can be achieved via electrical stimulation of culture medium via electrodes placed in cell culture dishes, causing the cells to contract at a set rate. This could easily be introduced into the systems, methods, and cell populations described herein (electrodes would be placed at either end of patterned surfaces such that the grooves ran between the electrodes). High concentrations of T3 hormone could also be introduced into the disclosed systems, methods, and cell populations. The patterned surfaces described herein are coated with fibronectin, but this could be replaced with various compounds, such as a polymer or matrix allowing modulation of either ECM composition (via introduction of different proteins) or stiffness (by changing the degree of crosslinking). Methods disclosed herein promote various maturation characteristics including cell elongation and anisotropy, sarcomere organization, and increased expression of markers of cell maturity. The disclosed methods and systems produce mature cardiomyocytes that possess characteristics that are not seen or seen less frequently in cell produced from other methods. The disclosed devices, methods, and systems are useful in producing cardiomyocyte cells that possess characteristics of maturity that are surprising in light of existing methods. Notably, the cells produced by the methods described here are ideal for use in modeling cardiovascular disease, while results described below indicate that cells cultured under standard, existing, conditions are much less so. In addition, the presently disclosed methods and systems produced cells with near adult-human-levels of myofibril tension generation, indicating a degree of advanced sarcomeric maturity in these cells that is not observed in cells produced from standard, existing, methods. The enhanced maturity of the presently disclosed cells was also observed via electron microscopy and assessment of sarcomere length. The presently disclosed methods and systems also produced cells which showed a highly elongated, rectangular morphology, with sarcomeres oriented perpendicular to the long axis of cells, which is consistent with the morphology of adult cardiomyocytes.

The disclosed devices, methods, and systems are useful in inducing a significantly enhanced hypertrophic response (increase in cell area and contractile response) in hiPSC-CMs subject to sustained culture, which is beneficial for using these cells for drug screening or disease modeling. Disclosed methods include identifying and isolating myofibrils from the resulting hiPSC-CMs, which allows for direct comparison of contractile function between the presently disclosed hiPSC-CMs and those obtained from human tissue. Remarkably, the maturation systems and methods disclosed herein increase force generation by the cells and improve kinetics of hiPSC-CM-derived myofibrils, shifting them towards an adult phenotype.

Hypertrophic cardiomyopathy (HCM) is a common form of cardiac dysfunction, affecting approximately one out of every 500 individuals. HCM may cause severe symptoms, loss of quality of life, and sudden death, yet is currently incurable, and the mechanisms by which this disease develops remain poorly understood. To further understand the mechanistic basis of HCM, myofibrils were isolated from human HCM heart explants and compared to control heart myofibrils—profound differences were found in both activation and relaxation kinetics in the HCM myofibrils. This is indicative of both contractile dysfunction and potentially diastolic dysfunction. Lines of hiPSC disclosed herein provide models for studying cardiomyopathy. By characterizing HCM on the sarcomeric level both in human tissue and in mature hiPSC-CMs, the mechanisms by which HCM develops, and how perturbations on the level of the myofibril lead to cell and organ-wide disease symptoms can be more easily studied.

### Induction Method

The hiPSCs may be induced to form cardiomyocytes by various methods. In some embodiments, induction is achieved by modulation of the Wnt signaling pathway, for example according to previously established methods known to those of skill in the art. In some embodiments, hiPSC-CMs may be plated into 24 well plates at a density of about 375,000 cells per well in mTESR1+Y-27632 (a ROCK inhibitor, Cayman Chemicals, Ann Arbor, Mich.). In some embodiments, the cells may be cultured in mTESR1 (STEMCELL Technologies, Vancouver, BC, CA) for about four days with daily media changes. In some embodiments this step may take about 3 to 6 days. In many embodiments, the media may then be changed to RPMI (RPMI) with B-27™ supplement without insulin (B-27-ins; 1:50, Life Technologies, Carlsbad, Calif.), and 8 μm CH IR99021 (Cayman Chemicals, Ann Arbor, Mich.), a GSK-3β inhibitor. In some embodiments, this may be referred to as day 0 (DO) in terms of cardiomyocyte (CM) age.

On day 1 of CM age, or about 24 hours later, the media may be replaced with RPMI/B-27-ins. 2 days later (D3), the media may be changed again, for example by combining old media (from D1) with fresh RPMI/B-27-ins, in a 1:1 ratio, supplemented with 5 μM IWP2 (Thermo Fisher Scientific, Waltham, Mass.), a Wnt inhibitor. In various embodiments, about 2 days later (D5), the media may be changed to RPMI/B-27-insulin. On D7, the media was replaced with RPMI with B-27 supplement with insulin (B27, 1:50, Life Technologies, Carlsbad, Calif.). The cells may then be maintained with RPMI/B27 with media changes every 3 days. In most cases, contracting cells are typically observed on D10-12 post induction, while large areas of plates typically began beating by D15-18.

### Maturity-Inducing Culture Medium

In most cases, upon observance of a robust cellular contraction in cultures of induced cardiomyocytes, the cells may be further induced to mature cardiomyocytes. In most embodiments, induced cardiomyocyte cells are digested using 0.25% Trypsin-EDTA (Thermo Fisher Scientific, Waltham, Mass.), pooled, and replated into 6 well plates at a ratio of about one 24-well plate well to about one 6-well plate well, and cultured in RPMI medium supplemented with Hyclone™ 20% FBS (GE Healthcare, Chicago, Ill.) and Y-27632. In most embodiments, the media may be changed after about 48 hours, to RPMI/B27. On D20-25 post-induction, cardiomyocytes may be selected for by various methods, including, for example the lactate method. In the lactate method, cells may be first washed with PBS, and the media changed to glucose-free DMEM supplemented with 4 mM lactate. These cells may be cultured in lactate media for about 5 days, with media changed on the third day of lactate culture. Media was then changed again to RPMI/B27. After about 3 days in RPMI/B27, the media may be changed to MM, for example media comprising palmitic and/or oleic acid. Switching the medium directly from lactate to MM, without 1-4 days in RPMI/B27, may result in cell death.

Disclosed systems, compositions, and methods include a maturity-inducing culture medium and methods of using same to induce maturation of cardiomyocytes. In many embodiments, the maturity-inducing culture medium include one or more fatty acids added to the media. In some embodiments, the fatty acid media may linoleic, palmitic acid and/or oleic acid, and the fatty acid may provide an energy source, for example one or more primary energy sources. The one or more fatty acids may be at various concentrations, for example from about 10 μM to about 200 μM. In many embodiments the fatty acid concentration in the medium is greater than about 10 μM, 20 μM, 30 μM, 40 μM, 50 μM, 60 μM, 70 μM, 80 μM, 90 μM, 100 μM, 110 μM, 120 μM, 130 μM, 140 μM, 150 μM, 160 μM, 170 μM, 180 μM, or 190 μM, and less than about 200 μM, 190 μM, 180 μM, 170 μM, 160 μM, 150 μM, 140 μM, 130 μM, 120 μM, 110 μM, 100 μM, 90 μM, 80 μM, 70 μM, 60 μM, 50 μM, 40 μM, 30 μM, 20 μM, or 10 μM. In many embodiments, the medium may further include a sugar, for example galactose, at a concentration between about 1 and 20 mM, for example 10 mM, and wherein the concentration is greater than about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, or 19 mM, and less than about 20 mM, 19 mM, 18 mM, 17 mM, 16 mM, 15 mM, 14 mM, 13 mM, 12 mM, 11 mM, 10 mM, 9 mM, 8 mM, 7 mM, 6 mM, 5 mM, 4 mM, 3 mM, or 2 mM.

The disclosed fatty acids may be bound to one or more compounds before adding to the medium. In some embodiments, the fatty acids may be bound or conjugated to a protein, such as albumin. Also disclosed are methods of binding or conjugating the disclosed fatty acids to a serum albumin protein, such as bovine serum albumin (BSA). In some embodiments, the disclosed conjugation may aid in introducing or adding the fatty acids into the disclosed culture medium.

### Patterned Cell Culture Surface

Disclosed systems, devices, and methods may include one or more patterned cell culture surfaces that aid in inducing cardiomyocyte maturation. In many embodiments, the disclosed device is a patterned cell culture surface, for example a cell culture surfaces including grooves, wherein the disclosed cells may adhere and grow. In some embodiments, the disclosed grooves may be formed in micro-patterned surfaces. The patterned cell culture surfaces may also include lapping paper and plastic coverslips and/or the use thereof. The use of lapping paper allows for producing fine, well aligned grooves on the coverslips—this technique has been shown to be consistent and reproducible in Applicant's experience. In other cases, existing methods of producing grooves on surfaces for use in cell culture typically require advanced, and costly, micro- or nanofabrication techniques. Thus, the disclosed method of producing grooves in growth surfaces is appealing in its simplicity and cost effectiveness. Lapping paper was purchased from McMaster-Carr (A4837A111). In many embodiments, the disclosed patterned growth surfaces may include 1 micron, 10 micron, 20 micron, 40 micron, or larger sized, lapping paper or the like. In many embodiments, the groves may have a width from from about 0.5 -1000 um, for example greater than about 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, or 95 μm, and less than about 100 μm, 90 μm, 80 μm, 70 μm, 60 μm, 50 μm, 40 μm, 35 μm, 30 μm, 25 μm, 20 μm, 19 μm, 18 μm, 17 μm, 16 μm, 15 μm, 14 μm, 13 μm, 12 μm, 11 μm, 10 μm, 9 μm, 8 μm, 7 μm, 6 μm, 5 μm, 4 μm, 3 μm, 2 μm, 1 μm, 0.9 μm, 0.8 μm, 0.7 μm, or 0.6 μm. In many embodiments, the disclosed growth surfaces may be coated with one or more compounds, molecules, and substances to improve cell attachment. In some embodiments, the one or more compounds, molecules, and substances may include a protein, for example a cell adhesion proteins. In one embodiment, the protein may be fibronectin, in another embodiment, collagen-based gelatin may be used. Matrigel® (Corning), which consists of an undefined mixture of extracellular matrix proteins, has also been used.

### Cells

Disclosed herein are cardiomyocyte cells with enhanced maturity. In many embodiments, the disclosed enhanced maturity cardiomyocytes are produced by the disclosed methods and systems, and may display many characteristics of adult human cardiomyocytes. In many embodiments, the characteristics may be selected from one or more of elongated cell morphology, enhanced maturity of sarcomeric structures, metabolic behavior, and increased myofibril contractile force. The presently disclosed enhanced maturity cardiomyocytes are hiPSC-CMs that may recapitulate pathological hypertrophy caused by one or more of a pro-hypertrophic agent or genetic mutations. In many embodiments, the disclosed cells may represent a suitable platform for modeling of hypertrophic disease and/or for research into drugs for treating various hypertrophies.

Disclosed herein are human induced pluripotent stem cell cardiomyocytes (hiPSC-CMs), and methods and systems for producing hiPSC-CMs that are more mature than existing in-vivo produced cardiomyocytes. In some embodiments, the disclosed hiPSC-CMs contain sarcomeres that are significantly larger and more organized that sarcomeres found in existing cardiomyocytes. In many embodiments, hiPSC-CMs produced by the methods and systems are elongated relative to other cells, for example GLUC and MM cells. In many embodiments, the disclosed cells have a degree of circularity that is less than cells produced in glucose media or grown in fatty acid media. In many embodiments, cells produced from these other methods (for example GLUC or MM) have a circularity that is, on average, greater than 1.2-fold, 1.3-fold, 1.4-fold, 1.5-fold, 1.6-fold, 1.7-fold, 1.8-fold, 1.9-fold, 2.0-fold, 3.0-fold, 4.0-fold, 5.0-fold, 6.0-fold, 7.0-fold, 8.0-fold, or 10.0-fold, and less than about 20.0-fold, 10.0-fold, 9.0-fold, 8.0-fold, 7.0-fold, 6.0-fold, 5.0-fold, 4.0-fold, 3.0-fold, 2.0-fold, 1.9-fold, 1.8-fold, 1.7-fold, 1.6-fold, 1.5-fold, 1.4-fold, 1.3-fold, 1.2-fold, or 1.1-fold that of the presently disclosed cells produced with the disclosed methods.

Sarcomeres in the presently disclosed cells and/or in cells produced from the disclosed methods and systems are elongated compared with cells produced from growth in glucose media alone (GLUC) or in MM. In some embodiments, the sarcomere has an average length of about 1.8 to 2.2 μm, for example greater than about 1.80 μm, 1.82 μm, 1.84 μm, 1.86 μm, 1.88 μm, 1.90 μm, 1.92 μm, 1.94 μm, 1.96 μm, 1.97 μm, 1.98 μm, 1.99 μm, 2.01 μm, 2.02 μm, 2.03 μm, 2.04 μm, 2.05 μm, 2.06 μm, 2.07 μm, 2.08 μm, 2.09 μm, 2.10 μm, 2.11 μm, 2.12 μm, 2.13 μm, 2.14 μm, 2.15 μm, 2.16 μm, 2.17 μm, 2.18 μm, 2.19 μm, or 2.20 μm, and less than about 2.25 μm, 2.20 μm, 2.19 μm, 2.18 μm, 2.17 μm, 2.16 μm, 2.15 μm, 2.14 μm, 2.13 μm, 2.12 μm, 2.11 μm, 2.10 μm, 2.09 μm, 2.08 μm, 2.07 μm, 2.06 μm, 2.05 μm, 2.04 μm, 2.03 μm, 2.02 μm, 2.01 μm, 2.00 μm, 1.99 μm, 1.98 μm, 1.97 μm, 1.96 μm, 1.95 μm, 1.94 μm, 1.93 μm, 1.92 μm, 1.91 μm, 1.90 μm, 1.88 μm, 1.86 μm, or 1.85 μm. In some embodiments the sarcomere length is, on average, greater than 10%, 11%, 12%, 13%, 14%, 15%, 16%, 17%, 18%, 19%, 21%, 22%, 23%, 24%, 25%, 26%, 27%, 28%, 29%, 30%, or 35%, and less than about 50%, 40%, 35%, 30%, 29%, 28%, 27%, 26%, 25%, 24%, 23%, 22%, 21%, 20%, 19%, 18%, 17%, 16%, 15%, 14%, 13%, 12%, 11%, or 10% than cells grown in glucose media (GLUC) alone or fatty acid media (MM) alone.

Myofibrils in the disclosed hiPSC-CMs may more closely mimic myofibrils from in-vivo tissue than myofibrils from cells produced by growth in glucose (GLUC) or fatty acid media (MM) alone. In some embodiments, the disclosed myofibrils may exert a maximum tension that is about 100% of that produced by myofibrils obtained from human tissue, for example greater than about 80%, 81%, 82%, 83%, 84%, 85%, 86%, 87%, 88%, 89%, 90%, 91%, 92%, 93%, 94%, 95%, 96%, 97%, 98%, 99%, 100%, or 105%, and less than about 110%, 99%, 98%, 97%, 96%, 95%, 94%, 93%, 92%, 91%, 90%, 89%, 88%, 87%, 86%, 85%, 84%, 83%, 82%, 81%, 80%, or 75%. In many embodiments, the disclosed myofibrils may exert a greater maximum tension than those in cells produced by growth in glucose (GLUC) or fatty acid (MM) alone, for example greater than about 1.0-fold, 1.2-fold, 1.3-fold, 1.4-fold, 1.5-fold, 1.6-fold, 1.7-fold, 1.8-fold, 1.9-fold, 2.0-fold, 3.0-fold, 4.0-fold, 5.0-fold, 6.0-fold, 7.0-fold, 8.0-fold, or 10.0-fold, and less than about 20.0-fold, 10.0-fold, 9.0-fold, 8.0-fold, 7.0-fold, 6.0-fold, 5.0-fold, 4.0-fold, 3.0-fold, 2.0-fold, 1.9-fold, 1.8-fold, 1.7-fold, 1.6-fold, 1.5-fold, 1.4-fold, 1.3-fold, 1.2-fold, or 1.1-fold.

### EXAMPLES

### Example 1—hiPSC-CM Differentiation and Culture

Disclosed herein are methods and systems for inducing hiPSCs into mature cardiomyocytes. In many embodiments, the disclosed methods may involve modulation of Wnt signaling, according to previously established methods. In some embodiments, 20-25 days after induction, the disclosed cardiomyocytes can be purified using lactate medium.

hiPSC-CMs may be cultured either in standard glucose-based media (RPMI 1640; referred to as ‘GLUC’), with maturation medium (MM) alone or with maturation medium and patterning (MPAT). In this example, hiPSC-CMs were cultured in RPMI-1640 (which contains 10.9 mM glucose) medium supplemented with B-27™ diluted 1:50 (i.e. GLUC) until approximately day 30 post induction. MM and MPAT cells were maintained in fatty acid/galactose-based maturation medium (containing 50 μM palmitic acid, 100 μM oleic acid, 10 mM galactose, and B-27™ supplement diluted 1:50). hiPSC-CMs were re-plated (either onto fibronectin-coated unpatterned or patterned surfaces or standard cell culture dishes) approximately day 40 post-induction. The patterned surfaces for the MPAT cells were prepared from clear plastic coverslips using 20 micron lapping paper, sterilized, and coated with fibronectin.

Compared to GLUC cells, both MM and MPAT hiPSC-CMs displayed a number of structural and functional improvements, including increased expression of fatty acid oxidizing genes, more mature mitochondria, and increased myofibril active tension generation. Additionally, while MM and MPAT cells displayed a robust, reversible response to the hypertrophic agonist phenylephrine, GLUC cells failed to do so, potentially indicating that prolonged culture in glucose-containing medium induces a hypertrophic state. hiPSC-CMs derived from patients with Danon disease cultured in MM or MPAT cells also displayed a robust hypertrophic response compared to GLUC. In particular, results indicated that the combinatorial approach employed for MPAT culture (i.e. fatty-acid containing media and patterned growth surfaces) produces hiPSC-CMs which demonstrate both adult-like myofibril mechanics and an adult-like hypertrophic response. Thus, use of the disclosed devices, methods and systems may be useful in studying various CM conditions associated with cardiovascular disease.

### Example 2—Maturation Methods Improve Both hiPSC-CM and Sarcomeric Morphology

The present disclosed combinatorial approach was also used to investigate whether hiPSC-CMs could be shifted towards more adult-like morphology and behavior. First, instead of using media where glucose is the main energy source (GLUC), cells were cultured in media where glucose was substituted with a combination of galactose, oleic acid, and palmitic acid (maturity medium, referred to as ‘MM’). Although both GLUC and MM each contain the B27™ supplement, which contains several fatty acids, MM provides ˜2700 times more total fatty acids than does media containing B27™ supplement alone. Second, MM-cultured hiPSC-CMs were plated onto plastic coverslips which had been micropatterned with grooves via lapping paper, to induce cell elongation and anisotropic alignment (MPAT). While previous studies have reported that use of commercially obtained nanopatterned grooved surfaces, specifically with a 700 nm groove size, may improve hiPSC-CM morphology, gene expression, and nucleation content, the presently disclosed methods used larger, more random, micropatterned surfaces to enhance the maturity level of induced hPSC-CMs. As disclosed above, the larger groove size was achieved by producing the grooved surfaces using 20 micron lapping paper.

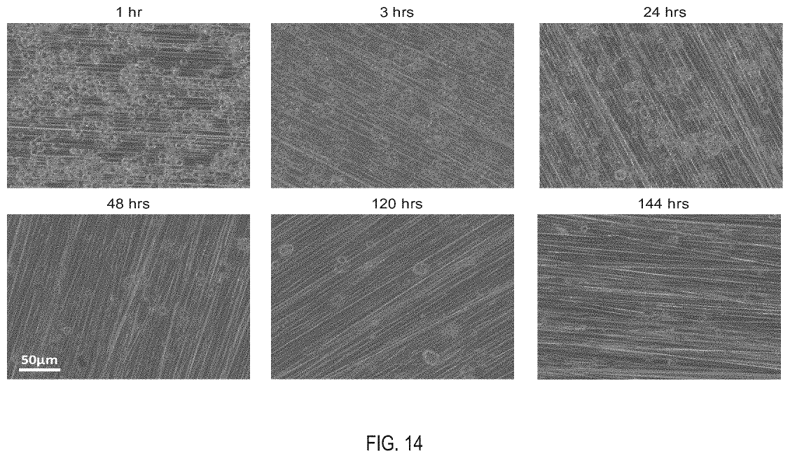

When plated onto the presently disclosed patterned growth surfaces, the disclosed cells adhered within hours, grew into the grooves within 24-48 hours (FIG. 14) and displayed coordinated uniaxial contraction along the direction of patterning, which was not observed in other conditions—for example with existing, commercial patterned surfaces.

Immunofluorescent microscopy was used to assess whether either patterning or MM induced changes in cellular morphology or sarcomere organization. Cells cultured under each condition expressed sarcomeric proteins cardiac troponin (cTnI), alpha actinin (α-act), ventricular myosin light chain 2 (MLC-2V), and myosin binding protein C3 (MYBPC3) (FIG. 8A). In MM and MPAT cells, the sarcomeres show greater levels of organization, with Z-lines oriented perpendicularly to the long axis of the cells, whereas in GLUC, sarcomeres wrapped around the cell in a circular fashion, or were oriented chaotically throughout the cytosol. Additionally, some GLUC cells lacked sarcomeres in significant areas of cytosol (FIG. 8A, white arrows), which was not observed in MM or MPAT cells.

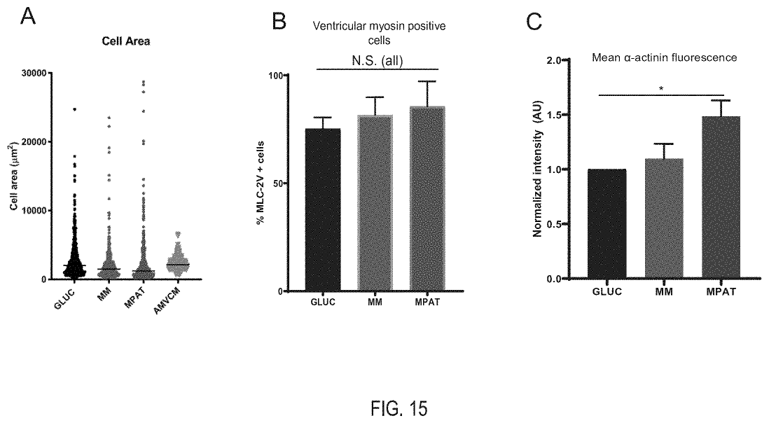

The different characteristics of the presently disclosed cells were also assessed by quantitative methods, for example measuring cell morphologies. Specifically, cells cultured with MM or MPAT were approximately 30% smaller than cells cultured in GLUC, consistent with previous reports (FIG. 8B). MM cells also showed a significantly lower circularity value than GLUC cells. Circularity was further reduced in MPAT cells, indicating that MPAT cells were significantly more elongated than MM cells, given their similar cell areas (FIG. 8C). While average cell area of GLUC cells was comparable to that of adult mouse cardiomyocytes, MM and MPAT cells were both significantly smaller, while all hiPSC-CM groups showed a significantly greater variability of cell area compared to adult mouse cells (FIG. 15A). All hiPSC-CM groups had cell areas lower than those reported for adult human cardiomyocytes (10-14,000 μm²). More than 75 percent of hiPSC-CMs cultured under all conditions stained positive for ventricular myosin light chain (MLC-2V) (FIG. 15B).

Using electron microscopy, sarcomeric morphology of hiPSC-CMs was examined. When cultured in standard glucose medium, hiPSC-CMs displayed chaotically aligned, disorganized sarcomeres, as well as Z-lines of varying thickness, which often did not pass through the entirety of sarcomeres, similar to what was observed using immunofluorescence (FIGS. 9A-B), and consistent with developing cardiomyocytes in the fetal heart. By contrast, MM cells displayed a more diverse sarcomere morphology: some sarcomeres were disordered, as seen in GLUC cells, whereas others were highly regular and organized, with I-bands, but lacking H-zones (FIGS. 9A-B). MPAT cells consistently displayed organized sarcomeres in which both I-bands and H-zones could be observed (FIGS. 9A-B), as is typically observed in more developed cardiomyocytes in vivo, or in more mature hiPSC-CMs.

As the heart develops, a greater proportion of cardiomyocyte volume becomes occupied by myofibrils, while average sarcomere length increases from approximately 1.6 to 2.2 μm. To assess whether changes in sarcomere occupancy were occurring in the presently disclosed MPAT cells, mean α-actinin fluorescence intensity was measured, as has been used previously. A significant (˜45%) increase in α-actinin fluorescence in MPAT cells, compared to GLUC cells, was observed (FIG. 15C). Sarcomere length was also seen to increase significantly, from ˜1.81 to 1.92 μm, from GLUC to MPAT (FIG. 9C) respectively. No significant increases in either α-actinin intensity or sarcomere length were observed between MM and GLUC conditions. This demonstrated that the presently disclosed combinatorial methods and systems, which result in the MPAT cells, induced cell maturity to the greatest degree.

### Example 3—Maturation Methods Improve Myofibril Force Generation

Since the presently disclosed devices, systems, and methods improved hiPSC-CM sarcomeric maturity and morphology, sarcomeric function was next investigated to determine whether it was correspondingly improved. In order to compare sarcomeric function of hiPSC-CMs to adult cardiac tissue, myofibrils were isolated from hiPSC-CMs cultured under each condition described herein. For comparison, myofibrils were also isolated from left ventricular tissue from a donor heart from a 35 year old male. Morphologically, striations, indicative of sarcomeres, could also be observed in some MPAT myofibrils, although not nearly to the extent of adult donor myofibrils, however, these structures were absent from most GLUC and MM hiPSC-CM myofibrils (FIG. 10A).

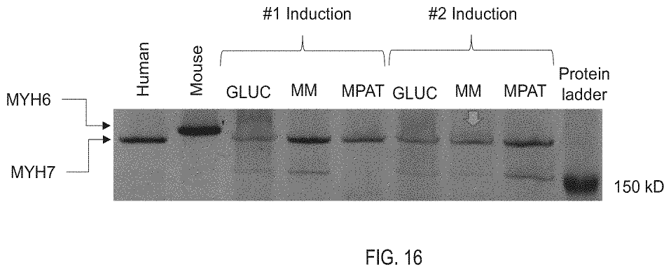

Remarkably, relative to GLUC, myofibrils from MPAT hiPSC-CMs demonstrated a nearly 150% increase in maximum tension generation, approaching the level of force generation measured the myofibrils of the adult donor heart (FIG. 100 and Table 1 below). MM myofibrils displayed an intermediate level of tension generation. Resting tension in GLUC, but not MM or MPAT myofibrils, was significantly lower than the resting tension from myofibrils isolated from donor hearts. Relaxation of myofibrils is biphasic, with a slow, linear phase, and a fast, exponential phase. Both slow and fast phase relaxation kinetics, and slow phase relaxation time, were similar between all hiPSC-CM-derived myofibrils and those from adult myocardial tissue. Unsupervised hierarchical clustering was also conducted on myofibril mechanical parameters in hiPSC-CMs and human myofibrils, and found that MPAT myofibrils clustered most closely with human adult myofibrils, again indicating increased maturity (FIG. 10B). To explore the mechanism behind increased MPAT force generation, expression of myosin heavy chain isoforms was examined, as well as of other myofibril-related genes on both the mRNA and protein levels. MPAT cells expressed predominantly MYH7 (FIG. 16A). On both the mRNA and protein levels, expression of most sarcomeric genes was quite similar across the different culture conditions. However, while MM and GLUC cells expressed both ventricular and atrial forms of myosin light chain (MLC-2V and MLC-2A), MPAT cells expressed primarily MLC-2V (FIG. 10D). While expression of non-muscle myosin (myosin II) was not detected in all hiPSC-CM samples, very low levels of slow skeletal troponin I was detected, which is typically expressed in the fetal heart. Taken together, these data further demonstrate that the presently disclosed devices, methods, and systems for creating MPAT cells induces hiPSC-CM maturity in myofibril mechanics.

TABLE 1
Myofibril mechanics data. *p<0.05 vs GLUC‡, p<0.05 vs
donor tissue: one way ANOVA followed by Tukey's multiple
comparison test on log2 transformed data.

| | GLUC | MM | MPAT | DONOR HRT |
|---|---|---|---|---|
| k_{REL},_{SLOW} (s⁻¹) | 0.20 ± 0.05^{‡} | 0.50 ± 0.14 | 0.44 ± 0.10 | 0.58 ± 0.09 |
| t_{LIN} (mSec) | 144.7 ± 11.2 | 139.6 ± 16.6 | 145.1 ± 13.7 | 167.9 ± 12.4 |
| k_{REL,FAST} (s⁻¹) | 8.88 ± 0.83 | 9.03 ± 1.21 | 9.51 ± 1.30 | 8.30 ± 0.95 |
| k_{ACT} (s⁻¹) | 1.04 ± 0.08 | 0.95 ± 0.10 | 1.07 ± 0.13 | 0.87 + 0.09 |
| k_{TR} (s⁻¹) | 0.87 ± 0.06^{‡} | 0.98 ± 0.07^{‡} | 0.76 ± 0.05 | 0.61 + 0.06 |
| Max. tens. (mN/mm²) | 21.40 ± 2.29^{‡} | 33.74 ± 5.49^{‡} | 52.10 ± 7.39^{*} | 67.16 ± 5.93 |
| Rest. tens. (mN/mm²) | 3.53 ± 0.4^{‡} | 6.49 ± 1.3^{‡} | 6.97 ± 1.13^{‡} | 11.98 + 2.14 |
| Diameter (μm) | 4.56 ± 0.22 | 4.00 ± 0.33 | 4.15 ± 0.36 | 5.46 ± 0.60 |
| N (# Myofibrils) | 30 | 24 | 31 | 13 |
| Age | N/A | N/A | N/A | 35 |

### Example 4—Maturation Induces a Gene Expression Program Controlling Fatty Acid Oxidation

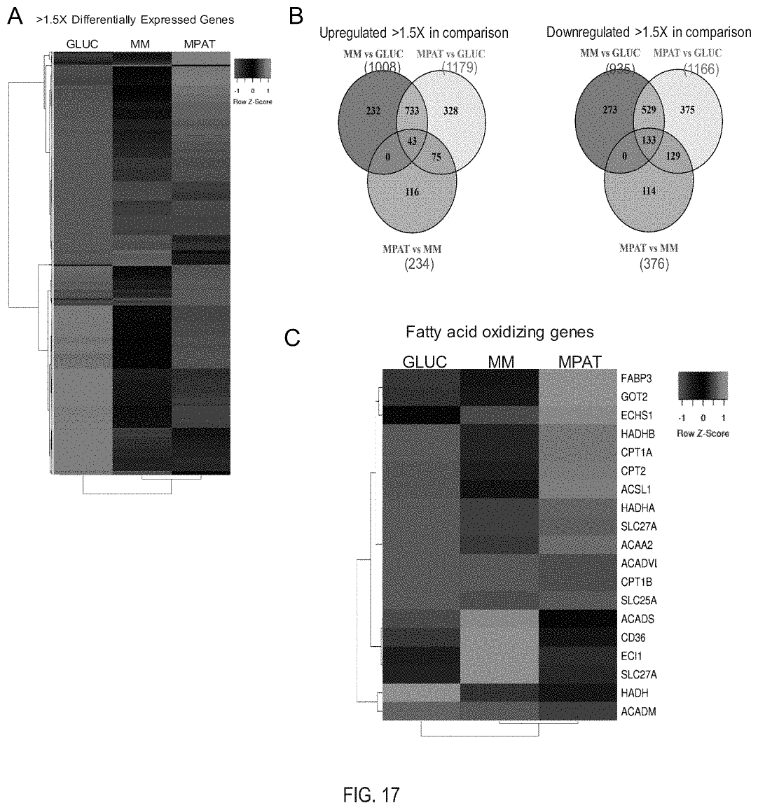

To further investigate mechanisms involved in enhancement of induced cardiomyocyte maturation by the presently disclosed devices, methods, and systems, RNA-seq was performed on hiPSC-CMs cultured under each condition. A large number of genes displayed differential expression between the GLUC and either MM or MPAT conditions, with a total of approximately 1000 genes showing either a 1.5-fold increase or decrease in expression (FIG. 17A-B). Most of the genes (733 upregulated and 529 downregulated genes) differentially regulated in either GLUC vs MM and GLUC vs MPAT were common between these two comparisons. Relatively few genes displayed differential expression between the MM and MPAT groups—only 234 genes were increased and 376 decreased by more than 1.5-fold (FIG. 17A-B). Gene ontology (GO) analysis was performed using PANTHER, and KEGG Pathway Analysis on differentially expressed genes. GO terms enriched in genes upregulated in MM or MPAT relative to GLUC hiPSC-CMs were frequently related to cardiac development or differentiation, or fatty acid metabolism, such as ‘regulation of heart morphogenesis’, ‘cardiocyte differentiation’, ‘fatty acid metabolism, and ‘fatty acid degradation’ (Tables 2.1-3.6 below). Tables 2.1-2.6 below show gene ontology on differentially expressed genes. RNA-seq data was filtered to include only top 12,500 expressed genes for all groups, then GO analysis was performed on genes displaying >1.5 fold regulation between designated groups. Tables 2.1-3.6 below show KEGG pathway analysis on differentially expressed genes. RNA-seq data was filtered to include only top 12,500 expressed genes for all groups, then KEGG Pathway analysis was performed on genes displaying >1.5 fold regulation between designated groups.

TABLE 2.1
↑ MPAT vs MM

| | Fold | | |
|---|---|---|---|
| GO Term | Enrichment | P value | FDR |
| regulation of multicellular organismal process | 1.94 | 5.88E-08 | 9.34E-04 |

TABLE 2.2
↓ MPAT vs MM

| | Fold | | |
|---|---|---|---|
| GO Term | Enrichment | P value | FDR |
| cell cycle | 4.08 | 5.00E − 32 | 7.95E − 28 |
| cell division | 6.67 | 3.26E − 29 | 2.59E − 25 |
| mitotic cell cycle | 5.3 | 2.43E − 27 | 9.64E − 24 |
| nuclear division | 7.81 | 1.09E − 21 | 2.88E − 18 |
| chromosome segregation | 7.77 | 4.23E − 21 | 9.61E − 18 |
| mitotic nuclear division | 11.4 | 2.40E − 20 | 4.24E − 17 |
| organelle fission | 7.07 | 2.56E − 20 | 4.07E − 17 |
| sister chromatid segregation | 10.93 | 1.32E − 18 | 1.90E − 15 |
| M phase | 9.20 | 1.61E − 17 | 2.14E − 14 |
| regulation of cell cycle | 3.19 | 4.21E − 17 | 4.78E − 14 |
| mitotic sister chromatid segregation | 12.23 | 5.76E − 17 | 6.11E − 14 |
| microtubule cytoskeleton organization involved in mitosis | 12.12 | 6.90E − 17 | 6.86E − 14 |
| spindle organization | 9.24 | 1.20E − 14 | 1.12E − 11 |
| biological phase | 6.27 | 1.30E − 14 | 1.15E − 11 |
| mitotic cell cycle phase | 6.27 | 1.30E − 14 | 1.09E − 11 |
| anaphase | 8.59 | 1.33E − 14 | 1.00E − 11 |
| chromosome organization | 3.12 | 2.92E − 14 | 2.02E − 11 |
| mitotic spindle organization | 13.4 | 3.81E − 14 | 2.52E − 11 |
| regulation of cell cycle process | 3.59 | 4.72E − 14 | 3.00E − 11 |
| mitotic prometaphase | 7.55 | 5.39E − 13 | 3.17E − 10 |
| regulation of mitotic nuclear division | 7.51 | 6.02E − 13 | 3.42E − 10 |
| microtubule-based process | 3.67 | 9.27E − 13 | 5.08E − 10 |
| regulation of chromosome segregation | 9.82 | 9.66E − 13 | 5.12E − 10 |
| regulation of mitotic sister chromatid separation | 13.51 | 4.75E − 12 | 2.44E − 09 |
| regulation of mitotic cell cycle | 3.59 | 6.45E − 12 | 3.21E − 09 |
| cell cycle | 4.08 | 5.00E − 32 | 7.95E − 28 |
| cell division | 6.67 | 3.26E − 29 | 2.59E − 25 |
| mitotic cell cycle | 5.3 | 2.43E − 27 | 9.64E − 24 |

TABLE 2.3
↑ MPAT vs GLUC

| | Fold | | |
|---|---|---|---|
| GO Term | Enrichment | P value | FDR |
| nervous system development | 1.72 | 1.97E − 14 | 3.13E − 10 |
| system development | 1.44 | 1.20E − 12 | 4.76E − 09 |
| anatomical structure development | 1.38 | 1.82E − 12 | 5.78E − 09 |
| developmental process | 1.35 | 7.84E − 12 | 2.08E − 08 |
| cell differentiation | 1.45 | 7.48E − 11 | 1.19E − 07 |
| regulation of multicellular organismal development | 1.63 | 4.12E − 10 | 5.04E − 07 |
| regulation of multicellular organismal process | 1.48 | 4.77E − 10 | 5.05E − 07 |
| anatomical structure morphogenesis | 1.6 | 7.18E − 10 | 7.14E − 07 |
| cell adhesion | 1.99 | 1.24E − 09 | 1.16E − 06 |
| axon development | 2.52 | 3.63E − 09 | 2.75E − 06 |
| cell morphogenesis involved in differentiation | 2.16 | 5.18E − 08 | 2.35E − 05 |
| cell projection morphogenesis | 2.19 | 1.42E − 07 | 5.25E − 05 |
| neuron projection morphogenesis | 2.19 | 1.94E − 07 | 7.01E − 05 |
| plasma membrane bounded cell projection | 2.17 | 2.24E − 07 | 7.93E − 05 |
| circulatory system development | 1.8 | 1.15E − 06 | 3.15E − 04 |
| regulation of localization | 1.4 | 1.29E − 06 | 3.41E − 04 |
| cardiovascular system development | 2.02 | 2.20E − 06 | 5.38E − 04 |
| plasma membrane bounded cell projection | 1.66 | 3.02E − 06 | 7.28E − 04 |
| organization | | | |
| mesenchyme morphogenesis | 4.85 | 1.38E − 05 | 2.37E − 03 |
| heart valve development | 4.28 | 2.18E − 05 | 3.28E − 03 |
| endocardial cushion development | 4.98 | 2.38E − 05 | 3.54E − 03 |
| heart valve morphogenesis | 4.31 | 7.97E − 05 | 9.83E − 03 |
| cardiac chamber morphogenesis | 2.79 | 1.57E − 04 | 1.67E − 02 |
| regulation of heart morphogenesis | 4.67 | 1.78E − 04 | 1.79E − 02 |
| mesenchymal cell differentiation | 2.6 | 1.81E − 04 | 1.81E − 02 |
| regulation of cholesterol biosynthetic process | 4.34 | 2.94E − 04 | 2.55E − 02 |
| regulation of lipid metabolic process | 1.87 | 3.13E − 04 | 2.60E − 02 |
| cardiac chamber development | 2.42 | 3.63E − 04 | 2.93E − 02 |

TABLE 2.4
↓ MPAT vs GLUC

| | Fold | | |
|---|---|---|---|
| GO Term | Enrichment | P value | FDR |
| cell cycle | 2.95 | 2.09E − 41 | 3.33E − 37 |
| mitotic cell cycle | 3.69 | 1.01E − 35 | 5.34E − 32 |
| cell division | 3.92 | 8.51E − 29 | 2.71E − 25 |
| nuclear division | 4.54 | 4.18E − 22 | 1.11E − 18 |
| organelle fission | 4.17 | 1.12E − 20 | 2.55E − 17 |
| chromosome segregation | 4.23 | 5.98E − 19 | 1.19E − 15 |
| DNA replication | 4.81 | 9.35E − 19 | 1.49E − 15 |
| chromosome organization | 2.38 | 9.44E − 19 | 1.37E − 15 |
| mitotic nuclear division | 5.9 | 1.16E − 18 | 1.54E − 15 |
| regulation of cell cycle | 2.26 | 2.36E − 18 | 2.89E − 15 |
| sister chromatid segregation | 5.8 | 1.15E − 17 | 1.30E − 14 |
| cytoskeleton organization | 2.26 | 8.07E − 17 | 8.55E − 14 |
| mitotic prometaphase | 5.04 | 8.12E − 17 | 8.07E − 14 |
| microtubule cytoskeleton organization | 3.07 | 3.35E − 16 | 2.96E − 13 |
| biological phase | 3.91 | 6.32E − 16 | 5.29E − 13 |
| mitotic cell cycle phase | 3.91 | 6.32E − 16 | 5.02E − 13 |
| organelle organization | 1.6 | 9.17E − 16 | 6.34E − 13 |
| cellular component organization | 1.42 | 1.35E − 15 | 8.93E − 13 |
| cell cycle phase transition | 3.73 | 2.12E − 15 | 1.35E − 12 |
| mitotic cell cycle phase transition | 3.75 | 2.86E − 15 | 1.75E − 12 |
| DNA conformation change | 3.65 | 4.65E − 15 | 2.74E − 12 |
| regulation of mitotic cell cycle | 2.57 | 4.62E − 14 | 2.53E − 11 |
| cellular component organization or biogenesis | 1.38 | 6.39E − 14 | 3.39E − 11 |
| microtubule-based process | 2.51 | 1.18E − 13 | 6.05E − 11 |
| DNA-dependent DNA replication | 5.17 | 1.06E − 12 | 5.12E − 10 |
| M phase (GO:0000279) | 4.27 | 1.57E − 12 | 7.36E − 10 |
| cell cycle | 2.95 | 2.09E − 41 | 3.33E − 37 |
| mitotic cell cycle | 3.69 | 1.01E − 35 | 5.34E − 32 |

TABLE 2.5
↑ MM vs GLUC

| | Fold | | |
|---|---|---|---|
| GO Term | Enrichment | P value | FDR |
| anatomical structure development | 1.49 | 4.32E − 17 | 6.87E − 13 |
| system development | 1.55 | 2.28E − 16 | 1.81E − 12 |
| cell differentiation | 1.55 | 1.37E − 13 | 3.11E − 10 |
| regulation of multicellular organismal | 1.81 | 1.67E − 13 | 3.32E − 10 |
| development | | | |
| cellular developmental process | 1.54 | 1.70E − 13 | 3.00E − 10 |
| cell adhesion | 2.29 | 3.47E − 13 | 5.01E − 10 |
| biological adhesion | 2.27 | 6.46E − 13 | 8.56E − 10 |
| anatomical structure morphogenesis | 1.74 | 2.69E − 12 | 3.28E − 09 |
| cell projection morphogenesis | 2.41 | 9.34E − 09 | 5.30E − 06 |
| circulatory system development | 1.97 | 3.56E − 08 | 1.38E − 05 |
| positive regulation of cell differentiation | 1.89 | 7.77E − 08 | 2.69E − 05 |
| cell projection organization | 1.78 | 1.63E − 07 | 4.55E − 05 |
| cardiovascular system development | 2.18 | 4.20E − 07 | 8.79E − 05 |
| regulation of cell projection organization | 2.01 | 5.68E − 07 | 1.13E − 04 |
| muscle structure development | 2.17 | 1.89E − 06 | 3.22E − 04 |
| regulation of lipid metabolic process | 2.18 | 9.06E − 06 | 1.29E − 03 |
| regulation of cholesterol metabolic process | 4.93 | 1.05E − 05 | 1.43E − 03 |
| muscle organ development | 2.38 | 1.84E − 05 | 2.31E − 03 |
| lipid metabolic process | 1.61 | 2.11E − 05 | 2.58E − 03 |
| response to glucocorticoid | 3.02 | 2.43E − 05 | 2.84E − 03 |
| response to growth factor | 1.96 | 2.50E − 05 | 2.90E − 03 |
| muscle tissue development | 2.28 | 4.34E − 05 | 4.72E − 03 |
| regulation of cholesterol biosynthetic process | 4.94 | 1.08E − 04 | 9.78E − 03 |
| regulation of sterol biosynthetic process | 4.94 | 1.08E − 04 | 9.72E − 03 |
| regulation of steroid metabolic process | 3.09 | 1.09E − 04 | 9.75E − 03 |
| striated muscle tissue development | 2.24 | 1.54E − 04 | 1.27E − 02 |
| cardiocyte differentiation | 2.93 | 2.91E − 04 | 2.11E − 02 |
| regulation of heart morphogenesis | 4.78 | 2.93E − 04 | 2.12E − 02 |

TABLE 2.6
↓ MM vs GLUC

| GO Term | P value | FDR |
|---|---|---|
| Cell cycle | 0.000033242 | 0.0052688 |
| Oocyte meiosis | 0.000033242 | 0.0052688 |
| Progesterone-mediated oocyte maturation | 0.00043059 | 0.045499 |
| ECM-receptor interaction | 0.00098342 | 0.077936 |
| Glycerolipid metabolism | 0.0014225 | 0.090184 |
| Glycine, serine and threonine metabolism | 0.0017477 | 0.092338 |
| cGMP-PKG signaling pathway | 0.0024853 | 0.11255 |
| Calcium signaling pathway | 0.0033015 | 0.13082 |
| Renin secretion | 0.0080179 | 0.28054 |
| Homologous recombination | 0.0091446 | 0.28054 |

TABLE 3.1
↑ MPAT vs MM

| KEGG Term | P value | FDR |
|---|---|---|
| Glycine, serine and threonine metabolism | 0.001581 | 0.50119 |
| ErbB signaling pathway | 0.0044073 | 0.53317 |
| Salivary secretion | 0.0056192 | 0.53317 |
| Axon guidance | 0.0067277 | 0.53317 |
| Adrenergic signaling in cardiomyocytes | 0.009867 | 0.62557 |
| PPAR signaling pathway | 0.014347 | 0.75802 |
| Phenylalanine metabolism | 0.01923 | 0.76198 |
| Selenocompound metabolism | 0.01923 | 0.76198 |
| Mineral absorption | 0.026744 | 0.94197 |
| Phosphatidylinositol signaling system | 0.036996 | 1 |

TABLE 3.2
↓ MPAT vs MM

| KEGG Term | P value | FDR |
|---|---|---|
| Cell cycle | 1.53E − 11 | 4.86E − 09 |
| Human T-cell leukemia virus 1 infection | 2.78E − 08 | 4.4127E − 06 |
| Cellular senescence | 9.2739E − 06 | 0.00097994 |
| p53 signaling pathway | 0.00092778 | 0.067806 |
| Hepatitis B | 0.0011578 | 0.067806 |
| Breast cancer | 0.0013546 | 0.067806 |
| Oocyte meiosis | 0.0014973 | 0.067806 |
| MAPK signaling pathway | 0.0019064 | 0.075543 |
| Colorectal cancer | 0.0026241 | 0.092425 |
| Small cell lung cancer | 0.0040755 | 0.12394 |

TABLE 3.3
↑ MPAT vs GLUC

| KEGG Term | P value | FDR |
|---|---|---|
| Fatty acid metabolism | 0.000010053 | 0.003187 |
| Fatty acid degradation | 0.00013291 | 0.021066 |
| Axon guidance | 0.00033531 | 0.035431 |
| Metabolic pathways | 0.00055519 | 0.043999 |
| AGE-RAGE signaling pathway in | 0.0012805 | 0.081183 |
| diabetic complications | | |
| Protein processing in endoplasmic | 0.0023937 | 0.1258 |
| reticulum | | |
| PPAR signaling pathway | 0.0027779 | 0.1258 |
| Glycosaminoglycan biosynthesis | 0.0043429 | 0.17209 |
| Fluid shear stress and atherosclerosis | 0.005151 | 0.18138 |
| Fatty acid elongation | 0.0059162 | 0.181 |

TABLE 3.4
↓ MPAT vs GLUC

| KEGG Term | P value | FDR |
|---|---|---|
| Cell cycle | 7.05E − 13 | 2.24E − 10 |
| Oocyte meiosis | 1.1788E − 06 | 0.00018683 |
| DNA replication | 2.5478E − 06 | 0.00026921 |
| Fanconi anemia pathway | 0.00003304 | 0.0023315 |
| p53 signaling pathway | 0.000036774 | 0.0023315 |
| Homologous recombination | 0.000064576 | 0.0034118 |
| Progesterone-mediated oocyte maturation | 0.00011021 | 0.0049911 |
| Cellular senescence | 0.0014981 | 0.059361 |
| Small cell lung cancer | 0.0019308 | 0.063087 |
| Base excision repair | 0.0019901 | 0.063087 |

TABLE 3.5
↑ MM vs GLUC

| KEGG Term | P value | FDR |
|---|---|---|
| Fatty acid metabolism | 0.00017398 | 0.044812 |
| Carbon metabolism | 0.00040047 | 0.044812 |
| Fatty acid degradation | 0.00042409 | 0.044812 |
| Metabolic pathways | 0.00083683 | 0.066319 |
| Fluid shear stress and atherosclerosis | 0.0010884 | 0.069008 |
| Cell adhesion molecules (CAMs) | 0.001613 | 0.074791 |
| Biosynthesis of amino acids | 0.0019203 | 0.074791 |
| MAPK signaling pathway | 0.00201 | 0.074791 |
| Alanine, aspartate and glutamate metabolism | 0.0021234 | 0.074791 |
| Steroid biosynthesis | 0.0026163 | 0.082938 |

TABLE 3.6
↓ MM vs GLUC

| KEGG Term | P value | FDR |
|---|---|---|
| Cell cycle | 0.000033242 | 0.0052688 |
| Oocyte meiosis | 0.000033242 | 0.0052688 |
| Progesterone-mediated oocyte maturation | 0.00043059 | 0.045499 |
| ECM-receptor interaction | 0.00098342 | 0.077936 |
| Glycerolipid metabolism | 0.0014225 | 0.090184 |
| Glycine, serine and threonine metabolism | 0.0017477 | 0.092338 |
| cGMP-PKG signaling pathway | 0.0024853 | 0.11255 |
| Calcium signaling pathway | 0.0033015 | 0.13082 |
| Renin secretion | 0.0080179 | 0.28054 |
| Homologous recombination | 0.0091446 | 0.28054 |

Several GO terms were also observed that were related to cellular process development and elongation in upregulated genes, which could be related to the elongated cellular morphology observed. Only a single GO term, ‘regulation of multicellular organismal process’ was enriched in MPAT relative to MM, while a few KEGG terms such as ‘adrenergic signaling in cardiomyocytes’ were enriched as well. By contrast, numerous GO and KEGG terms enriched in genes downregulated between MM versus GLUC or MPAT versus GLUC were related to DNA synthesis and cell division, including ‘cell cycle’, ‘cell division’, and ‘DNA replication’. Many of these terms were further downregulated in MPAT cells compared to MM. Without being limited by theory, this could indicate that culture in fatty acid medium is arresting cell cycle entry and cell division in MPAT hiPSC-CMs.

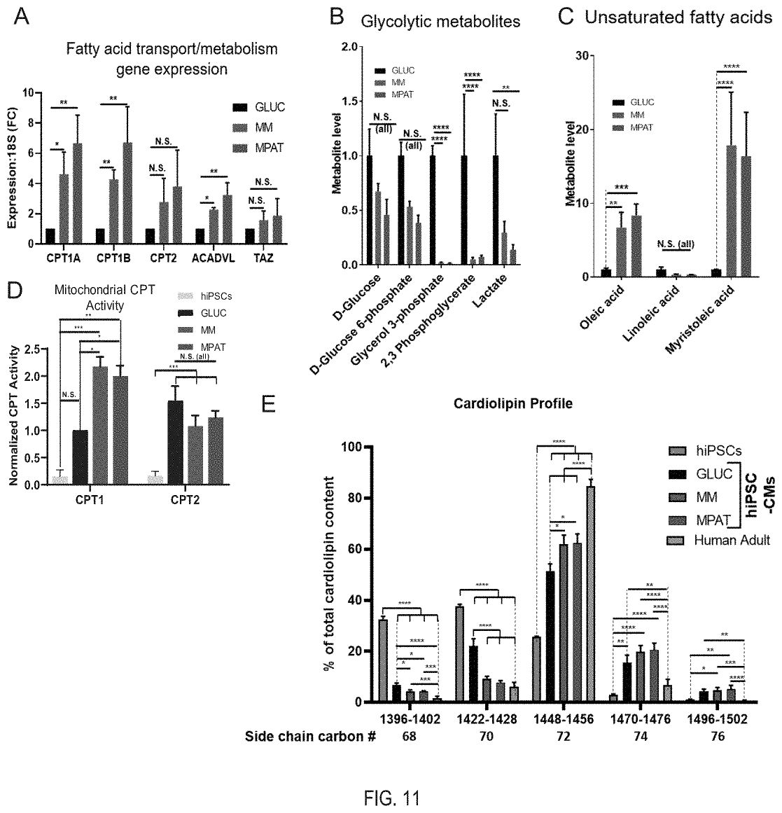

Based on the observed differences in GO terms related to cardiac development or fatty acid metabolism, changes in expression in genes involved in fatty acid metabolism were investigated in the RNA-seq data. In fact, many genes involved in mitochondrial fatty acid uptake and long chain fatty acid oxidation (such as CPT1A/1B/2 and ACADVL) were upregulated in MM, and further increased in MPAT hiPSC-CMs (FIG. 17C). To confirm these RNA-seq findings, expression of these targets was assessed using qPCR, and it was determined that several of these targets were significantly upregulated in MPAT cells, (FIG. 11A). Each of these targets also had higher average levels of expression in MPAT cells than MM cells.

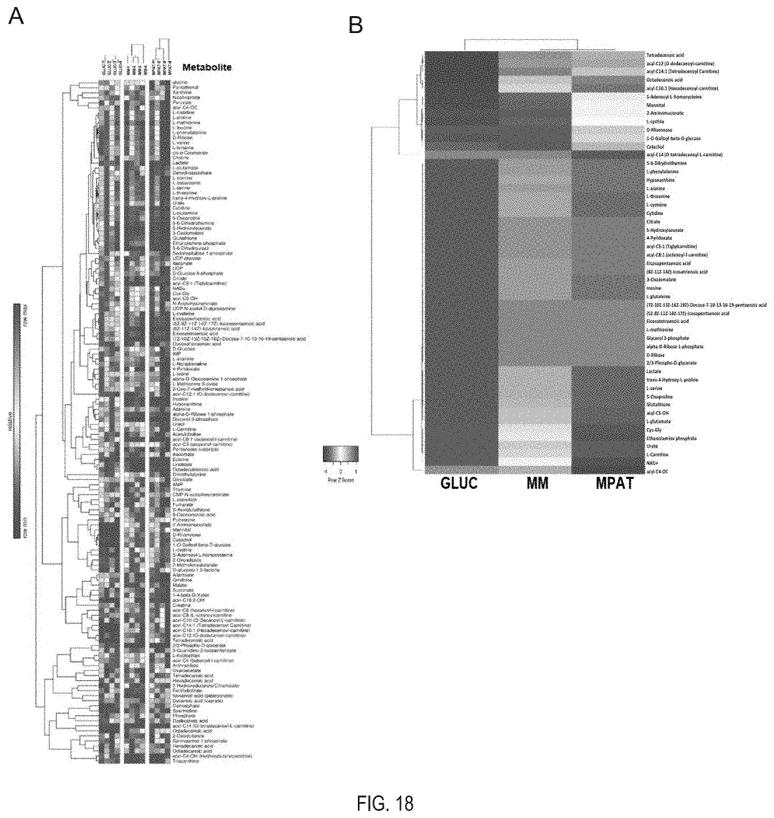

To further assess metabolic changes, metabolite screening was performed on hiPSC-CMs cultured under the described conditions. 136 polar metabolites were measured in high throughput profiling, of which 51 were significantly altered across the three conditions. Levels of most metabolites were lower in MM and MPAT hiPSC-CMs relative to GLUC (FIGS. 18A-B), while only 5 metabolites, acyl-C4-DC, acyl-C14, acyl-C14:1, catechol, and rhamnose, were significantly differentially produced when comparing MM and MPAT. Metabolites were further examined on a candidate basis, focusing on glucose and long chain fatty acid metabolism. Surprisingly, glucose levels in hiPSC-CMs cultured under each condition were not significantly different (FIG. 11B), although glucose was lacking from maturation medium. However, many metabolites of the glycolytic pathway were significantly reduced in MPAT hiPSC-CMs relative to GLUC. Levels of most saturated fatty acids were similar amongst each group of hiPSC-CMs, including palmitate, an ingredient in MM. By contrast, intracellular levels of oleic acid, myristoleic acid, and several medium or long chain acylated fatty acids, were much higher in MM and MPAT cells (FIGS. 11C, 18B). Taken together with the RNA-seq data, these results indicate that MPAT induces a shift from glycolysis to fatty acid oxidation as a source of energy production, as is seen in the developing heart. Without wishing to be limited by theory, the reduction in levels of glycolytic intermediates and products, and an increase in unsaturated fatty acids may be driving the structural and functional maturation observed in the MPAT cells.

### Example 5—Maturation Methods Enhance Mitochondrial CPT Activity and Cardiolipin Maturation

In the developing heart, the switch from glycolysis to fatty acid oxidation is accompanied with an increase in activity of the carnitine palmitoyltransferase (CPT) system. CPT1A/B/C acylate fatty acids, allowing their import into the mitochondria, where CPT2 then deacylates them to recover carnitine. Based on the observation of increased expression of the CPT genes by RNA-seq and qPCR, CPT activity was tested in hiPSC-CMs and in undifferentiated hiPSCs. Both CPT1 and CPT2 activity were profoundly increased in all hiPSC-CM groups relative to hiPSCs, while CPT1 was further increased in MM/M PAT groups (FIG. 11D). As CPT1 activity is considered to be the rate limiting step in long chain fatty acid oxidation, increased CPT1 activity is consistent with increased use of long chain fatty acids, and is likely indicative of metabolic maturation.

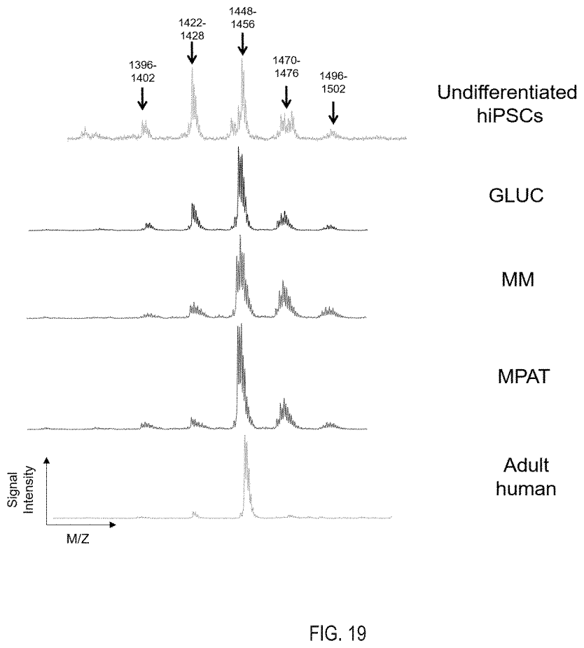

Cardiolipin (CL) is a critical component of mitochondrial membranes, and is essential for mitochondrial function. Each CL molecule has four fatty acid side chains. Side chain composition varies by tissue type, with the majority of CL in the adult heart having unsaturated fatty acid side chains, with tetralineolyl CL (total molecular weight of 1448 Daltons) by far the most abundant species. CL side chain composition is important for metabolic function in the heart, and defects in CL sidechain remodeling in the heart have been associated with Barth Syndrome. In the developing heart, cardiolipin content changes, with an increase in CL having side chains with 72 total carbons (72C-CL, MW 1448-1456) and a loss of most other species. Considering the metabolic improvements observed in the MPAT cells, the occurrence of CL remodeling was investigated in the more mature cells, using adult human hearts as control. Induction from hiPSCs into hiPSC-CMs caused significant CL remodeling, in particular a decrease in 68C- and 70C-CL, and an increase in 72C- and 74C-CL. With maturation methods, a further loss in 70C-CL and increases in both 72C- and 74C-CL were observed (FIGS. 11E and 19). These changes shifted the CL profile towards that of adult human hearts. Expression of Tafazzin (Taz), a major enzyme involved in CL remodeling in muscle tissues, was also assessed, but changes in its expression were not observed (FIG. 11A).

### Example 6—Maturation Methods Allow an Adult like Hypertrophic Response in hiPSC-CMs

The efficacy of maturation methods described herein was investigated for studying hypertrophic remodeling. Significant differences in the magnitude of the in vitro hypertrophic response have been observed in neonatal versus adult cardiac myocytes. In neonatal cardiomyocytes, agents such as the alpha-adrenergic receptor agonist phenylephrine, (PE) induce a 50-100% increase in cell area, whereas the response in adult cardiomyocytes is typically only 10-30%. Therefore, the response of hiPSC-CMs to PE was investigated. Since the BET-bromodomain inhibitor JQ-1 is known to inhibit cardiomyocyte hypertrophy both in vivo and in vitro; the ability of JQ-1 to block the effects of PE was tested in the disclosed system.

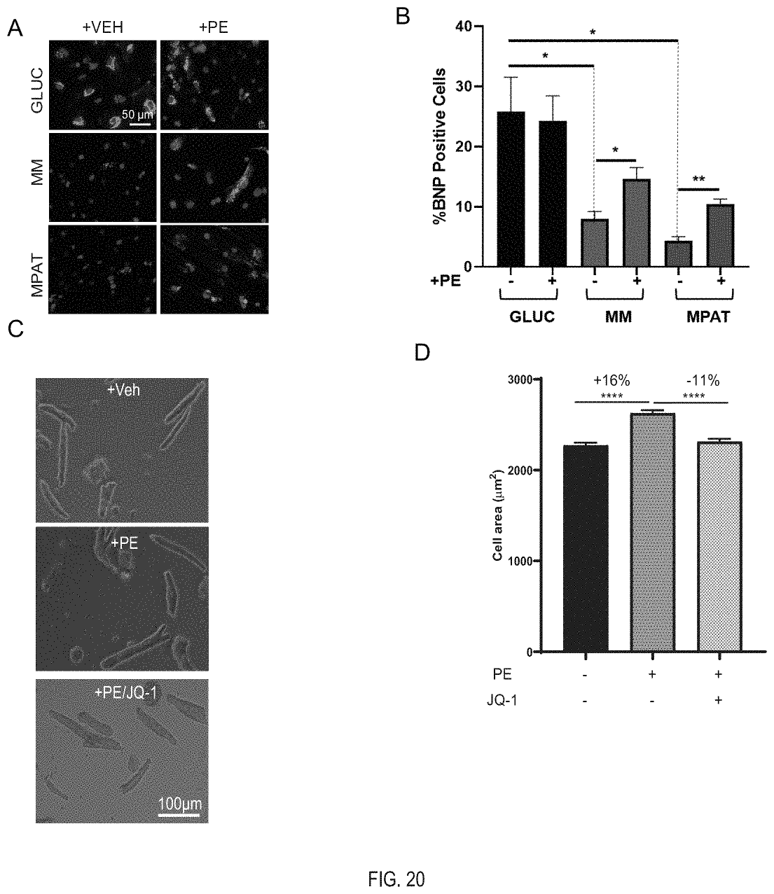

Surprisingly, PE treatment had no effect on cell area of hiPSC-CMs cultured in glucose-containing medium, yet JQ-1 treatment nonetheless profoundly reduced cell area (FIGS. 12A-B). By contrast, in hiPSC-CMs cultured in improved maturation conditions, PE treatment induced a strong hypertrophic response. Specifically, PE treatment led to a 29% increase in cell area for MM and a 19% increase for MPAT cells, respectively. In both groups, this hypertrophy was blocked by JQ-1 treatment: cell area relative to PE-treated cells was reduced by 31% in MPAT and 17% in MM cells. To validate these findings, expression of NPPB (BNP), a robust marker of hiPSC-CM hypertrophy, was investigated by qPCR and immunofluorescence. By qPCR, MM and MPAT cells displayed an approximately 5-6 fold reduction in BNP expression relative to GLUC cells (FIG. 12C). Furthermore, more than 20% of GLUC cells stained positive for BNP expression, which was not increased by PE treatment. Basal BNP staining in MM and MPAT cells was much lower, yet was increased 2 to 2.5 fold by PE treatment (FIGS. 20A-B). Taken together, these results suggest that prolonged glucose culture may be inducing hiPSC-CM hypertrophy, and that proper selection of culture conditions may be essential to characterizing hypertrophic cardiomyopathy using the hiPSC-CM platform.

To compare MPAT hiPSC-CMs directly to adult cells, adult mouse ventricular cardiomyocytes (AMVCMS) were isolated and treated with PE and JQ-1. In AMVCMS, PE induced a 16% increase in cell area, while JQ-1 induced an 11% decrease (FIGS. 20C-D).

Having demonstrated that disclosed maturation methods and systems allow for induction of an agonist-based hypertrophic response in hiPSC-CMs, cardiac hypertrophy was investigated to determine if it could be characterized in patient-specific cells. In previous experiments, Applicants generated multiple lines of hiPSC-CMs from Danon disease patients carrying mutations in the LAMP2 gene, leading to pathological cardiac remodeling. Interestingly, echocardiography indicated that the patients from whom the hiPSC-CMs were generated displayed varying degrees of cardiac hypertrophy and dysfunction: thus, hiPSC-CMs from two patients, MD-186 and MD-111, were focused on. These patients had moderate and severe cardiac hypertrophy, respectively. The left ventricular posterior wall thickness (LVPW) of patient MD-111 was 1.8 cm, whereas the LVPW of MD-186 was 1.0 cm. Compared to previously reported LVPW values in healthy adult males (0.6-1.0 cm, mean 0.8 cm), these values represent a 125% and 20% increase, respectively. When cultured in GLUC media, both sets of Danon-derived hiPSC-CMs displayed a modest and similar increase in cell area (30-40% increase), compared to hiPSC-CMs derived from MF750, a healthy male donor (FIG. 12D-E). However, when cultured in MPAT or MM, the two Danon cell lines displayed differential degrees of hypertrophy: MD-111 hiPSC-CMs displayed extreme hypertrophy (138% increase in MPAT), whereas the hypertrophy of MD-186 cells was comparatively modest (66% increase in MPAT). The extent of hypertrophic remodeling observed in these two cell lines under the presently disclosed maturation methods and systems correlated relatively well to the increase in left ventricular posterior wall thickness (LVPW) observed in the hearts of patients MD-186 and MD-111. Thus, the presently disclosed methods and systems are useful in generating more mature cells that recapitulate patient-specific phenotypes.

### Example 7—PE Treatment of Mature hiPSC-CMs Induces Myofibril Relaxation Changes as Observed in Hypertrophic Cardiomyopathy

Previous reports have indicated that myofibrils isolated from hearts of humans with hypertrophic cardiomyopathy (HCM) display profound differences in mechanical behavior. Applicants therefore examined whether myofibrils isolated from hypertrophic hiPSC-CMs might demonstrate similar myofibril mechanical perturbations. Myofibril mechanics were assessed in multiple tissue bank samples of human donor and HCM hearts. Specifically, myofibrils were isolated from 4 unused donor hearts and 4 hearts from patients with hypertrophic cardiomyopathy (HCM) with a left ventricular posterior wall thickness of >12 mm as assessed by echocardiography and received transplants. All myofibril mechanical parameters from donor and HCM hearts are listed in Table 4 below. Compared to donor hearts, HCM hearts-derived myofibrils showed a shortened linear phase relaxation time, as well as faster activation kinetics (FIG. 13A). These results are similar to findings from myofibrils isolated from a human heart with an HCM-causing mutation in MYH7.

TABLE 4
Human Donor and HCM Myofibril Characteristics

| | Donor HRT | HCM HRT |
|---|---|---|
| k_{REL, SLOW} (s⁻¹) | 0.45 ± 0.09 | 0.83 ± 0.25 |
| t_{LIN} (mSec) | 157.1 ± 3.2 | 114.6 ± 15.3* |
| k_{REL, FAST} (s⁻¹) | 8.53 ± 0.50 | 10.82 ± 3.19 |
| k_{ACT} (s⁻¹) | 0.78 ± 0.16 | 1.38 ± 0.17* |
| k_{TR} (s⁻¹) | 0.53 ± 0.04 | 0.74 ± 0.05** |
| Max. tens. (mN/mm²) | 53.55 ± 12.61 | 47.81 ± 5.03 |
| Rest. tens. (mN/mm²) | 11.50 ± 2.83 | 8.03 ± 2.33 |
| Diameter (μm) | 5.62 ± 0.37 | 4.43 ± 0.15* |
| N (# hrts) | 4 | 4 |
| Age | 31.25 ± 0.48 | 47.5 ± 3.92* |
| Sex (# male, # female) | 4:0 | 3:1 |

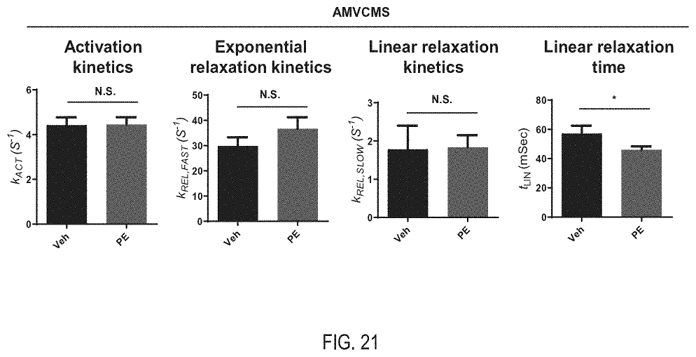

Myofibrils were then isolated from hypertrophic hiPSC-CMs. For this experiment, myofibrils were isolated from MPAT hiPSC-CMs only, as these displayed the most adult-like myofibril behavior. Hypertrophy was again induced via treatment with PE. To compare hiPSC-CMs to hypertrophic adult cardiomyocytes, myofibrils were also isolated from vehicle or PE-treated AMVCMs. Remarkably, myofibril relaxation was shown to be altered in the PE-treated hiPSC-CMs—the exponential phase relaxation constant was increased, while the duration of linear phase relaxation was shortened (indicating faster relaxation) (FIG. 13B). Activation kinetics also trended towards an increase. In AMVCMs, PE induced similar relaxation changes, but no changes in activation kinetics or could be detected (FIG. 21A). PE treatment therefore induced similar changes in myofibril relaxation as is observed in human HCM. Together, these results indicated that the MPAT hiPSC-CM platform is a suitable in vitro model for human hypertrophic cardiomyopathy, allowing parallel assessment of changes in cell area, hypertrophic gene expression, and myofibril mechanical behavior.

### Materials and Methods

hiPSC-CM Differentiation: hiPSCs were induced into cardiomyocytes via modulation of Wnt signaling, according to previously established methods. Specifically, hiPSC-CMs were plated into 24 well plates at a density of 375,000 cells per well. Cells were plated in mTESR1+Y-27632 (a ROCK inhibitor, Cayman Chemicals, Ann Arbor, Mich.), and were then cultured in mTESR1 (STEMCELL Technologies, Vancouver, BC, CA) for four days with daily media changes. After four days, media was changed to RPMI (RPMI) with B-27™ supplement without insulin (B-27-ins; 1:50, Life Technologies, Carlsbad, Calif.), and 8 μm CHIR99021 (Cayman Chemicals, Ann Arbor, Mich.), a GSK-3β inhibitor. This was considered day 0 (D0) in terms of cardiomyocyte age. Exactly 24 hours later, media was replaced with RPMI/B-27-ins. 2 days later (D3), media was changed again: at this point, old media (from D1) was combined with fresh RPMI/B-27-ins, in a 1:1 ratio, supplemented with 5 μM IWP2 (Thermo Fisher Scientific, Waltham, Mass.), a Wnt inhibitor. 2 days later (D5), media was changed to RPMI/B-27-ins. On D7, media was replaced with RPMI with B-27 supplement with insulin (B27, 1:50, Life Technologies, Carlsbad, Calif.). Cells were then maintained with RPMI/B27 with media changes every 3 days. Contracting cells were typically observed on D10-12 post induction, while large areas of plates typically began beating by D15-18.

Once robust cellular contraction was observed in most plates, cells were digested using 0.25% Trypsin-EDTA (Thermo Fisher Scientific, Waltham, Mass.), pooled, and replated into 6 well plates at a ratio of 1-24 well plate well to 1-6 well plate well, in RPMI medium supplemented with Hyclone™ 20% FBS (GE Healthcare, Chicago, Ill.) and Y-27632. Media was changed after 48 hours, to RPMI/B27. On D20-25 post induction, cardiomyocytes were selected for using the lactate method: cells were washed with PBS, then media was changed to glucose-free DMEM supplemented with 4 mM lactate. Cells were cultured in lactate media for 5 days, with media changed on the third day of lactate culture. Media was then changed again to RPMI/B27. In the case of cells cultured under MM or M PAT conditions, media was changed to MM after 3 days in RPMI/B27. Switching medium directly from lactate to MM appeared to cause significant levels of cell death so was avoided.

Patterned surface preparation: Patterned surfaces were created by drawing 20 micro lapping paper across 22 mm×22 mm plastic coverslips (VWR International, Radnor, Pa., Cat. No. 48376-049), 20-30 times per coverslip. Coverslips were held in place during patterning to ensure consistency of direction of the grooves created by the patterning process. Surfaces were then washed in soapy water to remove plastic dust or other contaminants, and soaked repeatedly in ultrapure water. For imaging studies, coverslips were cut into 11 mm×11 mm squares. To sterilize, surfaces were incubated overnight in 100% ethanol. The following day, surfaces were placed in 6 well plates, and residual ethanol allowed to evaporate. Surfaces were then incubated under cell culture ultraviolet for 10-15 minutes for sterilization. Fibronectin solution in PBS (6 μg/mL, Corning Inc, Corning, N.Y.) was then added to each surface, and fibronectin coating allowed to occur at room temperature for 1-2 hours. Surfaces were then washed in PBS, and allowed to completely dry before adding cells to surfaces. For imaging studies, GLUC and MM hiPSC-CMs were cultured on unpatterned surfaces which were prepared in an identical fashion to patterned surfaces except for the lapping paper step. For other experiments, GLUC and MM cells were cultured on fibronectin-coated 6 well plate wells.

Cardiomyocyte replating onto surfaces: On day 35-40 post induction, cardiomyocytes were replated onto patterned surfaces. Cells were digested for 7-9 minutes using Accumax (Innovative Cell Technologies, San Diego, Calif.) at 37° C., then digestion was halted by addition of RPMI-20. Cells were counted, then pelleted by centrifuging at 1000 g for 3 minutes, then resuspended in RPMI-20 supplemented with Y-27632. When plating onto large (2 cm×2 cm) patterned surfaces, 600-750,000 cardiomyocytes were plated per surface in 500-750 μL cultured medium, which was sufficient to create a confluent, uniformly beating cell sheet. When plating onto small (8 mm×8 mm) patterned or unpatterned surfaces for imaging studies, 15,000 cells were plated per surface. Cell suspension was added dropwise onto each surface; cell medium stayed on the surfaces via surface tension. (This step is conducted very carefully to avoid cell solution running off the surface—it is useful to ensure that the surface is completely dry before adding cell suspension). Cells were then allowed to attach to the surface at 37° C. for 45 minutes before adding an additional 1.5 mL RPMI-20+Y-27832 medium. Medium was changed to MM after 48 hours, and then changed biweekly thereafter.

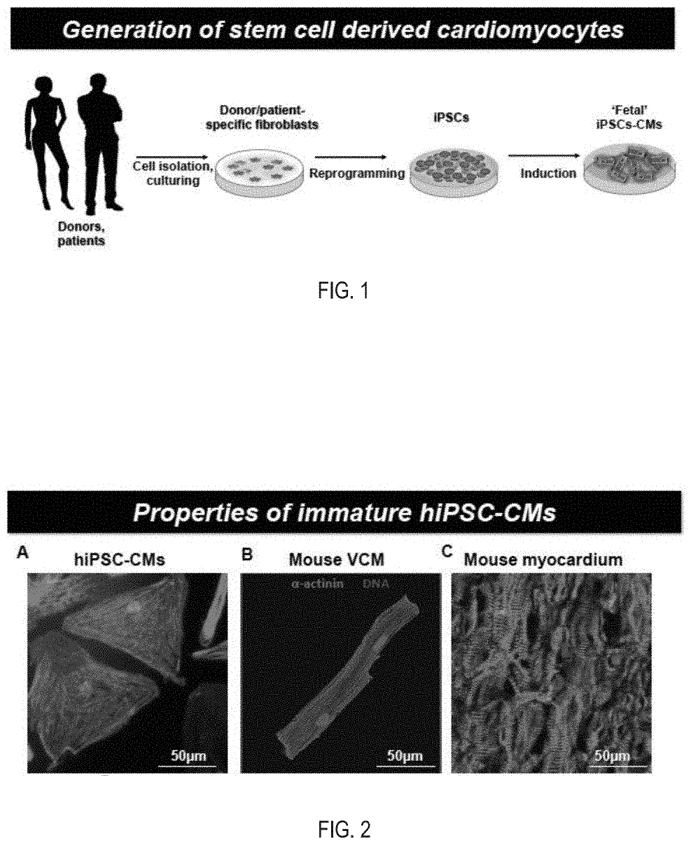

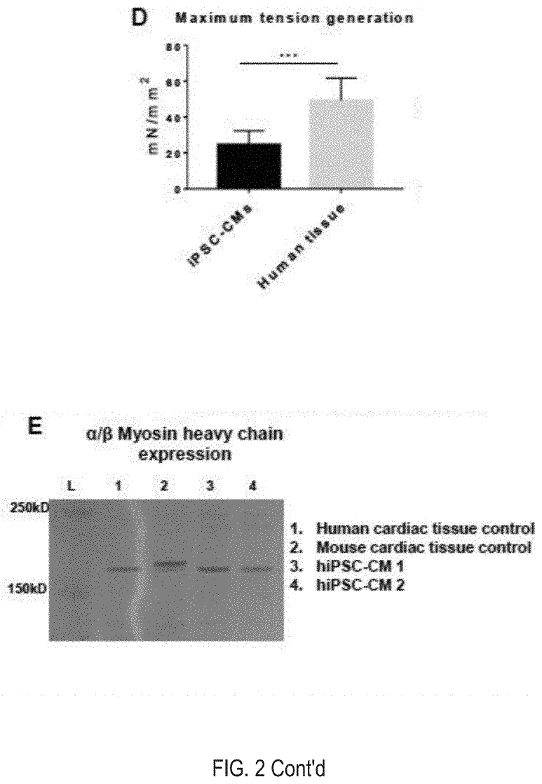

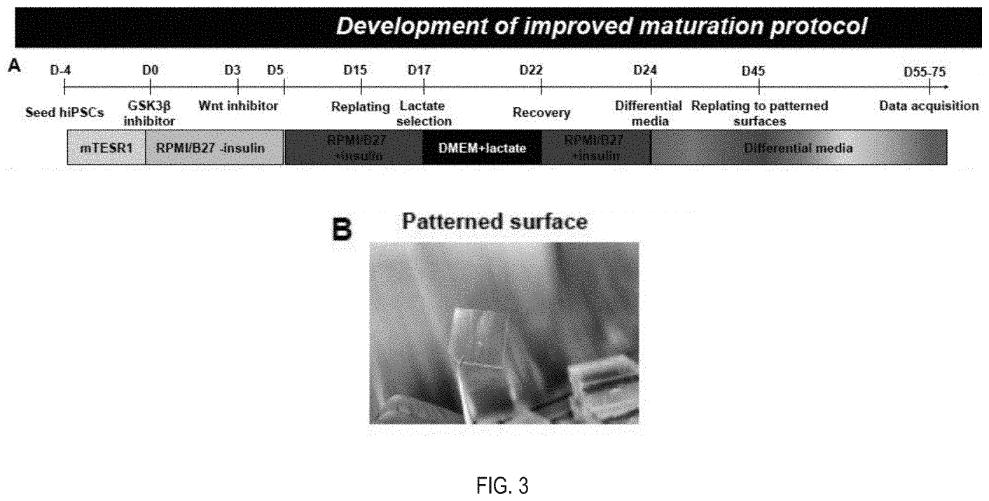

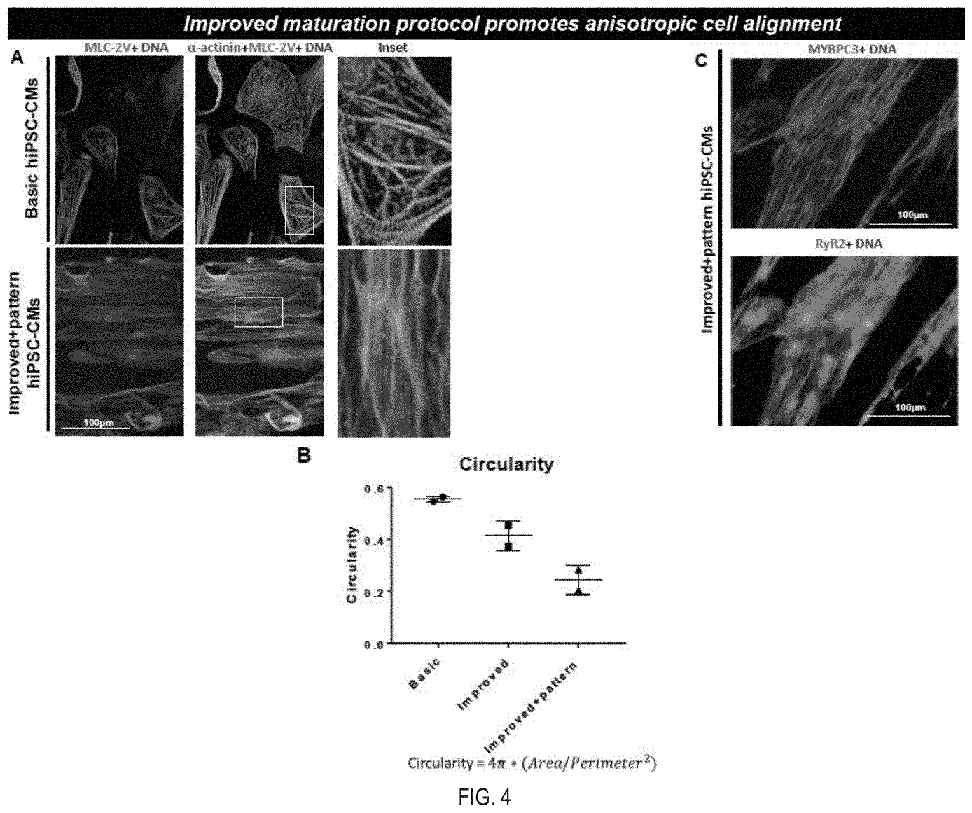

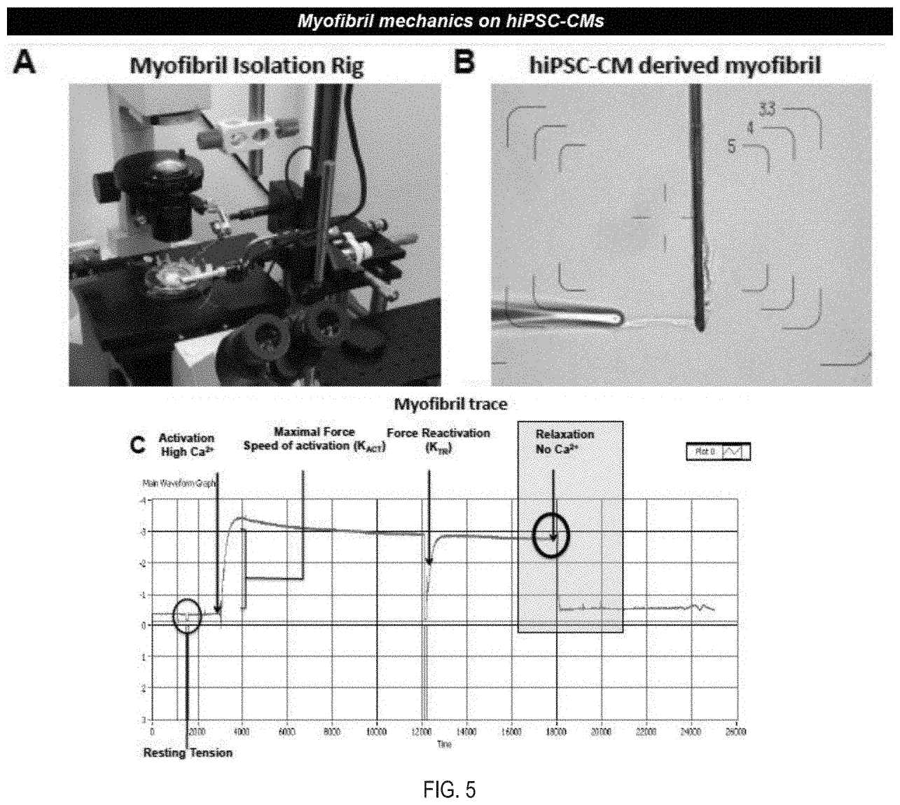

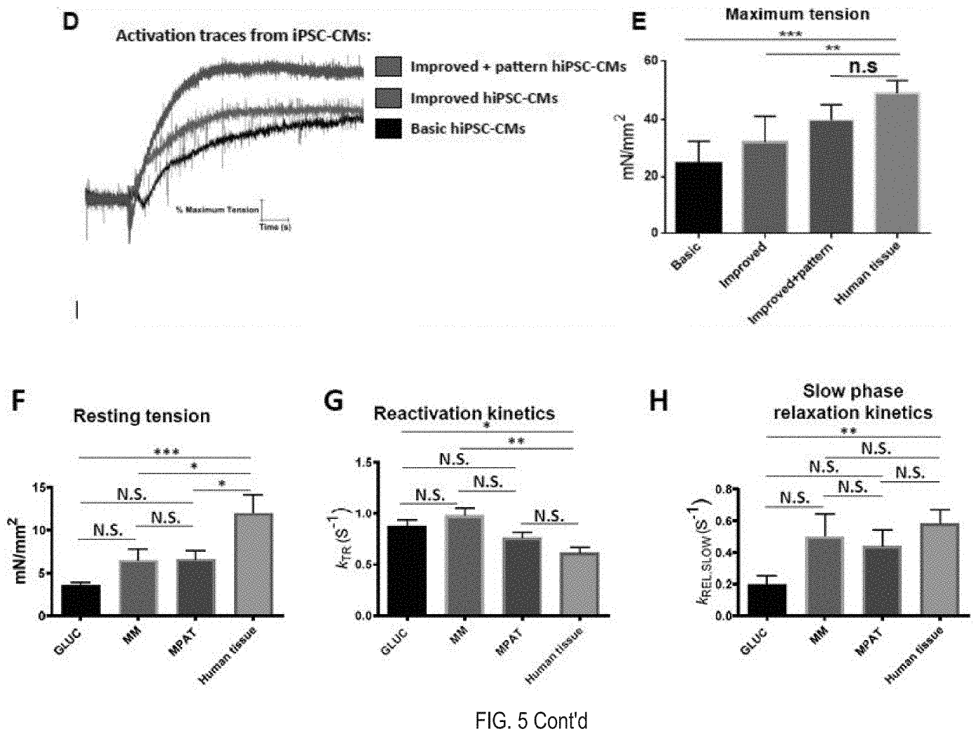

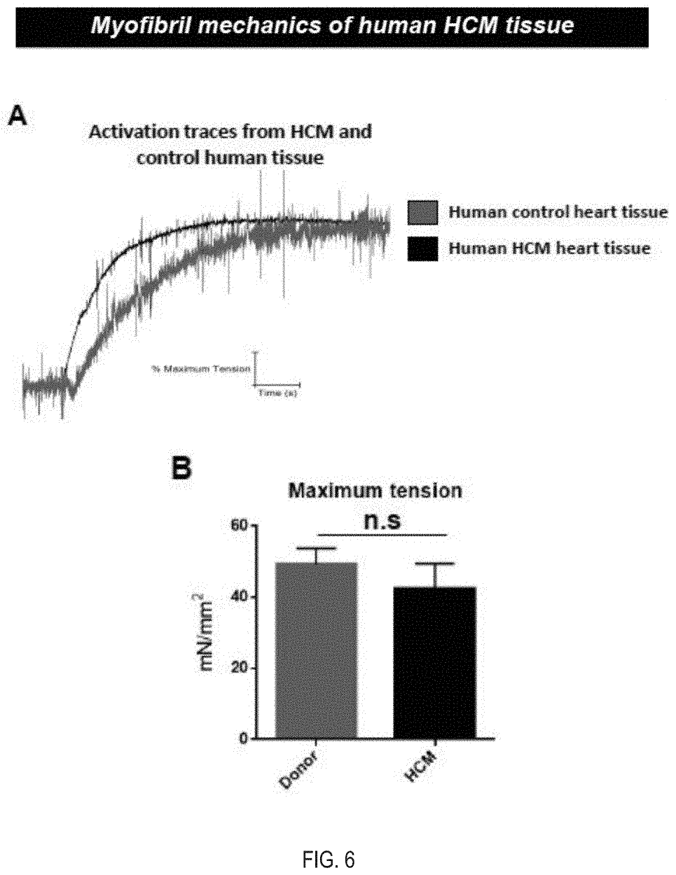

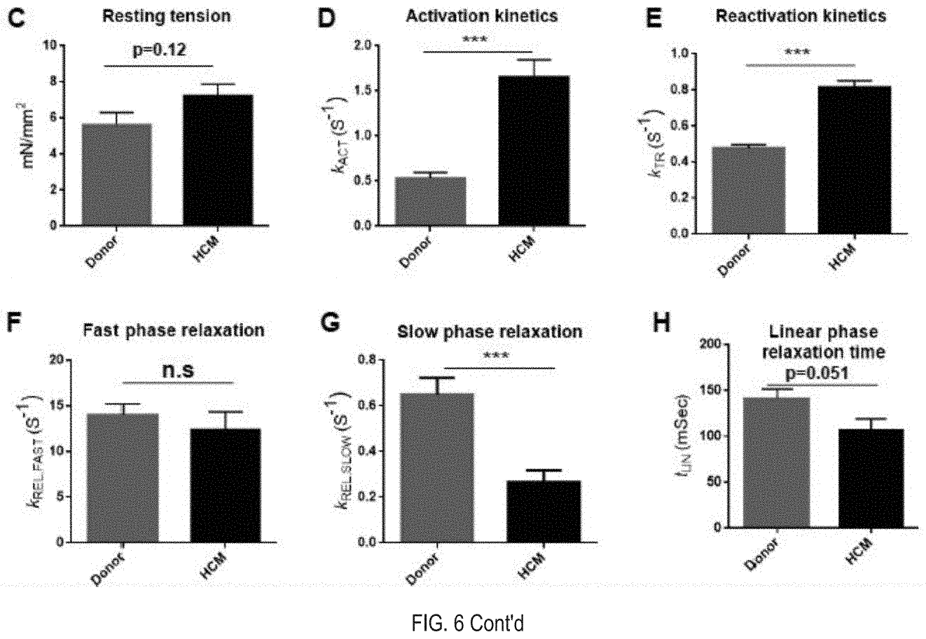

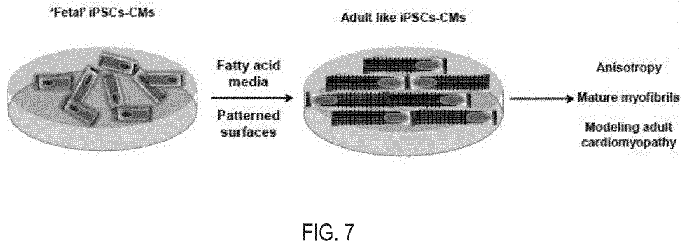

Cell imaging studies: Cells on small unpatterned or patterned surfaces in 24 well plates were used for immunofluorescent staining. Briefly, cells were washed with PBS, then fixed either with 4% paraformaldehyde (PFA) in PBS (10 minutes at room temperature) or a 1:1 mixture of methanol and acetone (5minutes at -20° C.). After fixation, cells were washed with PBS, then permeabilized with 0.2% Triton-X 100 in PBS (10 minutes at room temperature). Methanol/acetone fixation was used for images shown in FIG. 1 (staining for α-actinin, MYBPC3, c-TNI, and MLC-2V). PFA fixation was used for all other experiments. All measurements of cell area and hypertrophy were conducted on PFA fixed cells.

For immunofluorescent staining, after permeabilization, cells were treated with blocking buffer (10% horse serum in PBS) for 30 minutes at room temperature. Cells were then treated with primary antibodies diluted in 5% horse serum/PBS for 2 hours, washed four times in PBS for 5 minutes, and treated with secondary antibodies in 5% horse serum/PBS for 1 hour, washed twice with PBS for 5 minutes, counterstained with Hoescht 3342 in PBS for 5 minutes, and washed twice with PBS for 5 minutes, all at room temperature.

For cell area measurements, cells were PFA fixed and stained for α-actinin as described above, then counterstained with HCS CellMask™ Orange (Thermo Fisher Scientific, Waltham, Mass.), diluted 1:2, 500 in PBS, for 30 minutes at room temperature.

For B-type natriuretic peptide (BNP) staining, an adaption of a previously published method was used: cells were PFA fixed, then blocked and permeabilized for 30 minutes with 5% milk/0.1% Triton X-100/PBS (blocking solution) for 30 minutes at room temperature. Cells were then incubated with pro-BNP antibody diluted in blocking solution overnight at 4° C. Cells were then washed four times in 0.05% Triton-X 100/PBS for 5 minutes, and incubated with secondary antibody diluted in blocking solution for hour at room temperature. Cells were then washed twice in 0.05% Triton-X 100/PBS for 5 minutes, counterstained with Hoescht 3342 as described above, and washed twice more.

All imaging was conducted on an EVOS FL Cell Imaging System (Thermo Fisher Scientific, Waltham, MA). All image analysis including measurements of cell area, circularity, and sarcomere length were conducted using Image J.

For immunostaining, the following primary antibodies were used: 1) Ventricular myosin light chain 2 (MLC-2v): Proteintech 10906-1AP rabbit antibody at 1:400 (Proteintech Group, Rosemont, Ill.); 2) Alpha actinin: Sigma-Aldrich A7811 mouse antibody at 1:400 (MilliporeSigma, Burlington, Mass.) ; 3) Myosin binding protein C3: Santa Cruz E-7 sc-137180 mouse antibody at 1:400 (Santa Cruz Biotechnology, Inc., Dallas, Tex.); 4) Cardiac troponin I: Phosphosolutions 2010-TNI rabbit antibody at 1:200 (PhosphoSolutions, Aurora, Colo.); 5) B-type natriuretic peptide (and pro-nt-BNP): Abcam ab13115 [15F11] mouse antibody at 1:200 (Abcam, Cambridge, UK). The following secondary antibodies were used: 1) Alexa Fluor 488 goat anti-rabbit A11034; 2) Alexa Fluor 546 goat anti-rabbit A21428; 3) Alexa Fluor 488 A11029 goat anti-mouse; 4) Alexa Fluor goat anti-mouse 546 A21422 (Thermo Fisher Scientific, Waltham, Mass.). All secondary antibodies were used at 1:400 dilution.

MYH6/7 measurements: Prior to lysis, cells were detached by treatment with Accumax, counted, and pelleted. Pellets were then lysed by resuspending pellets in isoelectric focusing buffer (IEF) with protease inhibitors. For human and mouse heart samples, approximately 20 mg of powdered left ventricular tissue per sample was resuspended in IEF by vortexing, then spun at 5000 g for 5 minutes at 4° C. to remove insoluble tissue. To determine protein content, a 10 μL aliquot of each lysate (diluted as necessary) was pH neutralized by the addition of 40 μL 0.12N HCl, then protein concentration was measured by Bradford Assay on these neutralized samples and compared to a standard curve to calculate protein concentrations. 10 μg of protein per sample was loaded onto a degassed 6% SDS-PAGE gel with the following modifications: resolving gel buffer pH at 9.0; running gel buffer pH at 8.2; inner gel buffer supplemented with β-mercaptoethanol at 600 μL/L; and separating acrylamide/bis ratio 1:100. These gels were run overnight at 0.12V at 4° C., and stained with modified Coomassie blue/silver solution to stain protein.

Immunoblotting: Prior to lysis, cells were detached by treatment with Accumax, counted, and pelleted. Pellets were then lysed by resuspending pellets in ice-cold lysis buffer (150 mM NaCl, 50 mM Tris-Cl pH 7.4, 1 mM EDTA, 1% Triton, with Complete mini tablet (Roche, Basel, CH), and 1 mM phenylmethylsulphonyl fluoride freshly added before use). Protein concentrations were determined using a BCA assay according to manufacturer's instructions (Bio-Rad Laboratories, Hercules, Calif.). 4 μg of protein per sample was loaded onto a precast 4-12% Criterion Tris-HCL gel (Bio-Rad Laboratories, Hercules, Calif.). Protein was transferred overnight onto activated polyvinylidene fluoride (PVDF) membrane overnight at 0.12V at 4° C. For Western blotting, membranes were blocked for 1 hour with 5% milk/TBST buffer for 1 hour at room temperature, incubated with primary antibody in TBST buffer overnight at 4° C., and incubated in secondary antibody in 5% milk/TBST buffer for 1 hour at room temperature, with TBST washes between antibodies. The following primary antibodies were used for Western blotting: 1) Ventricular myosin light chain 2 (MLC-2v): Proteintech 10906-1AP rabbit antibody at 1:1000; 2) Atrial myosin light chain 2 (MLC-2a): Proteintech 17283-1-AP rabbit antibody at 1:1000 (Proteintech Group, Rosemont, Ill.); 3) Myosin binding protein C3: Epitomics 3153-1 rabbit antibody at 1:1500 (Epitomics, Inc, Burlingame, Calif.); 4) Alpha actinin: Sigma A7811 mouse antibody at 1:2000 (MilliporeSigma, Burlington, Mass.); 5) Cardiac/slow skeletal troponin I: Abcam Ab10239 [M F4] at 1:1:1000 (Abcam, Cambridge, UK, note that while this antibody is listed as cardiac specific, the epitope it was raised against is common to cardiac and slow skeletal TNI); 6) Non-muscle myosin (Myosin IIB): Sigma M7939 rabbit antibody at 1:1000 (MilliporeSigma, Burlington, Mass.); 7) alpha tubulin: Cell Signaling #3873 mouse antibody at 1:5000 (Cell Signaling Technology, Danvers, Mass.). The following secondary antibodies were used: 1) For mouse primary antibodies: Southern Biotech goat anti-mouse 1031-05, at 1:2000-1:5000 (for alpha tubulin, was used at 1:5000, Southern Biotech, Birmingham, Ala.); 2) For rabbit primary antibodies: Sigma A9169 at 1:2000-1:5000 (MilliporeSigma, Burlington, Mass.). Membranes were developed using Supersignal™ Pico Plus (Thermo Fisher Scientific, Waltham, Mass.).

Metabolomic Screening: Prior to sample submission, cells were detached by treatment with Accumax, counted, and pelleted, and flash frozen using liquid nitrogen. 131,000-875,000 cells were submitted per individual sample, with samples submitted from four independent hiPSC-CM inductions. Metabolites from frozen cell pellets were extracted using ice-cold methanol/acetonitrile/water (5:3:2) at a ratio of 2e6 cells per mL by vortexing for 30 minutes at 4° C. Samples were clarified through centrifugation (10 minutes at 10,000 rpm, 4° C.) and 10 μL of supernatant was analyzed using a 5 minute C18 gradient on a Thermo Vanquish UHPLC coupled online to a Thermo Q Exactive mass spectrometer operating in positive and negative ion modes (separate runs) as previously described in detail. Metabolites were assigned using Maven (Princeton University) in conjunction with the KEGG database. Assignments and quality control were performed as previously described.

Cardiolipin Quantification: Cardiolipin was quantified using previously published methods with normal phase liquid chromatography coupled to electrospray ionization mass spectrometry in an API 4000 mass spectrometer (Sciex, Framingham, Mass.). To perform this assay with hiPSC-CMs, cells were dissociated using Accumax and counted, with 1-7.5×10⁵ cells from each condition were harvested for the assay. Cells were flash frozen in liquid nitrogen prior to use. Lipids were extracted using a modified Bligh Dyer method according to previously published methods with 1000 nmoles tetramyristal cardiolipin as an internal standard (Avanti Polar Lipids, Alabaster, Ala.).

Carnitine Palmitoyl Transferase Activity Assay: Rates of CPT1 and CPT2 were quantified using a 14C carnitine based radioactive assay. The assay measures the activity of CPT1 by permeabilizing the plasma membrane and measuring the production of palmitoyl carnitine from palmitoyl CoA. By permeabilizing the mitochondrial inner membrane and adding malonyl CoA to inhibit CPT1, the activity of CPT2 was measured. To perform this assay with iPSC cells, cells were dissociated using Accumax and counted, with 1.75-7.5×10⁵ cells from each condition were harvested for the assay. CPT activity was normalized to cell counts.

RNA extraction and cDNA generation: RNA was generated via a column-free RNA extraction method. Cells were initially detached and counted with Accumax, and were then lysed in 1 mL TRIzol™ (Thermo Fisher Scientific, Waltham, Mass.) reagent per cell pellet. 250 μL chloroform was then added to each sample, mixed by vortexing, and centrifuged at 12,500 rpm for 15 minutes at 4° C. Upper phase was then decanted, and an equal volume of isopropanol and 2 μL glycogen to each RNA solution. Samples were centrifuged again at 12,500 rpm for 20 minutes at 4° C., and supernatants decanted from RNA pellet. Pellets were washed with 70% ethanol/ultrapure DNAase/RNAase-free H₂O, and centrifuged again at 12,500 rpm for 5 minutes at 4° C. RNA pellets were then resuspended in 17 μL ultrapure DNAase/RNAase-free H₂O per pellet.

Genomic DNA was removed via DNAase I treatment. RNA was then recrystallized via addition of a 100 mM LiCl₂/74% (vol:vol) EtOH/2% (vol:vol) glycogen solution to the RNA solution. This solution was then incubated at —80° C. for 90 minutes; RNA was then pelleted by centrifugation at 12,500 rpm for 30 minutes at 4° C. Pellets were then washed once with an ice cold 70% (vol:vol) ethanol solution, and centrifuged again at 12,500 rpm, and resuspended in 20 μL RNAase/DNAase-free H₂O. RNA yield was determined using the Nanodrop™ (Thermo Fisher Scientific, Waltham, Mass.). cDNA was generated using the iScript cDNA Kit (Bio-Rad) according to manufacturer's instruction, using 250 ng-1 μg RNA as substrate, using a mixture of random primers. qPCR gene expression analysis was performed using SYBR Green Supermix (Bio-Rad), according to manufacturer's instructions, using 0.5 μL cDNA in a 20 μL total reaction volume. Each reaction was performed in duplicate.

The following primer sets were used for qPCR analysis:

### Primer Sequences

| | | |
|---|---|---|
| Human | for | 5′-TGC TTC ACA GTG GAG CTG ATA-3′ |
| TNNI3 | rev | 5′-GCT GCA ATA TGC AAT GGA GTG-3′ |
| Human | for | 5′-CGC CAA CTC CAA CGT GTT CT-3′ |
| MYL2 | rev | 5′-CCA TCC CTG TTC TGG TCC AT-3′ |
| Human | for | 5′-CTG CTG CTT TGG TGT CAG AG-3′ |
| ACTN2 | rev | 5′-TTC CTA TGG GGT CAT CCT TG-3′ |
| Human | for | 5′-GAC TCC TCT GAT CGA TCT GC-3′ |
| NPPA | rev | 5′-GTT ATC TTC AGT ACC GGA AGC-3′ |
| Human | for | 5′-TCC TGC TCT TCT TGC ATC TGG-3′ |
| NPPB | rev | 5′-TTT GCC CTG CAA ATG GTT-3′ |
| Human | for | 5′-CCT GGA AGG TGA CAG ATG AAT G-3′ |
| ACADVL | rev | 5′-CCC TCA AAG ATC CGG AAG ATG-3′ |
| Human | for | 5′-CAC ACA CAG TGG AAT GGA AAT G-3′ |
| CPT1A | rev | 5′-CCT GCT TGG ATG ATG CTA AAT G-3′ |
| Human | for | 5′-GCT ATG TGT ATC CGC CTT CTA TC-3′ |
| CPT1B | rev | 5′-GAC TCT AGG TAC CGC TGA ATT G-3′ |
| Human | for | 5′-GCT TTG ACC GAC ACT TGT TTG-3′ |
| CPT2 | rev | 5′-TGT GGT TTA TCT GCC CGT ATG-3′ |
| Human | for | 5′-GAG AAC AAG TCG GCT GTG G-3′ |
| TAZ | rev | 5′-GCT GGA GGT GGT TGT GGA-3′ |
| Human | for | 5′-GCC GCT AGA GGT GAA ATT CTT A-3′ |
| 18S rRNA | rev | 5′-CTT TCG CTC TGG TCC GTC TT-3′ |

RNA-seg protocol: Total RNA was prepared as described above. Prior to submission, RNA quality was confirmed via analysis on Tapestation (Agilent Technologies, Santa Clara, Calif.). Library preparation and RNA-seq was performed by BGI Genomics (Shenzhen, CN). Briefly, mRNA was purified from total RNA using oligo(dT) magnetic beads, then fragmented. First strand cDNA was generated using random hexamers, followed by second strand synthesis. cDNA was then subjected end repair, and 3′-adenylation, adapter ligation, PCR amplification, and circularization, creating a single strand circle DNA library. RNA-seq was performed at a read depth of 40 million reads, using paired-end sequencing, on the DNBSeq Platform.

Quality control of the RNA-seq data was performed with FastQC45. The RNAseq reads were mapped to human GRCh38 reference genome with the splice-aware STAR aligner v2.7. The read counts were generated from the aligned reads with featureCounts function in the RsubRead package. The read counts were normalized with DESeq2 R package.

Gene Ontology and Pathway Analysis: Gene ontology analysis was performed using PANTHER and KEGG. Prior to analysis, RNA-seq data was filtered to only include to top 15,000 most highly expressed genes by total expression (across the GLUC, MM and M PAT conditions). Data were then sorted by fold change between each 2 condition comparison. These comparisons were filtered for genes showing either a 1.5× upregulation or downregulation between groups in question, and these filtered data fed separately into PANTHER for gene ontology analysis, or WebGestalt for KEGG Pathway analysis. For Panther, ‘Statistical overrepresentation test’ was selected, using ‘GO biological process, complete’, and the Homo sapiens whole-genome list as reference. For WebGestalt, ‘Overrepresentation analysis’ was also selected, using ‘pathway/KEGG’, a minimum of 10 genes per category, and the Homo sapiens whole genome list as reference.

Cardiomyocyte Isolation: 15 minutes prior to beginning isolation, mice were injected with 300 μL of heparin (10,000 units/mL). Mice were then anesthetized via inhalation of isofluorane, and when becoming unresponsive to toe pinch were killed via cervical dislocation. The chest was then opened, and the heart was removed rapidly and placed in ice-cold perfusion buffer (10 mM creatine monohydrate, 30 mM Taurine, 5.6 mM Dglucose, 4.6 mM NaHCO₃, 10 mM BDM, 120 mM NaCl, 15 mM KCl, 0.6 mM Na₂HPO4, 0.6 mM KH₂PO4, 1.2 mM MgSO₄, 10 mM Hepes, filtered at 0.40 μm, pH 7.4). The heart was washed briefly in perfusion buffer, and excess fat/thymus tissue trimmed off. The aorta was then cannulated using a blunted 20-gauge needle, and was mounted on a Langendorff apparatus (Radnoti, Covina, Calif.) with a heating pump kept at 37° C. for the duration of the isolation (VWR International, Radnor, Pa.). Hearts were mounted on the Langendorff apparatus within 3-7 minutes of removing from the body. The heart was then perfused in retrograde fashion at a rate of 4 mL/minute, driven by a Rainin™ Rabbit peristaltic pump (Mettler Toledo, Columbus, Ohio). To remove blood from the heart's vessels, it was first perfused for 3 min using perfusion buffer. Buffer was then switched to calcium-free digestion buffer (perfusion buffer with 1.3 mg/mL collagenase II) for 3 min and was then switched to digestion buffer supplemented with 28 nM CaCl₂. Hearts were perfused with high-calcium digestion buffer for another 6 to 10 minutes, once the heart became extremely soft and compliant. The heart was then removed from the cannulating needle and placed in a 60 mm culture dish with 2.5 mL high-calcium digestion buffer. The atria and right ventricle were then removed, and the left ventricle dissociated using a pipette and forceps. Once the heart was completely dissociated, digestion was halted by the addition of 7.5 mL stopping buffer (perfusion buffer with 10% (vol/vol) FBS and 12.5 nM CaCl₂). The cardiomyocyte suspension was then filtered through 200-μm mesh into a 50-mL conical tube, and the culture dish washed with an additional 10 mL stopping buffer, which was then filtered into the conical tube as well. This cell suspension was then allowed to incubate for 10 minutes at room temperature, allowing a cardiomyocyterich pellet to form. The fibroblast-rich supernatant was discarded, while the pellet with was resuspended in 10 mL of stopping buffer. 100 mM CaCl₂ solution was then added to this buffer in a stepwise manner, with 2-min intervals between each addition: calcium concentration was first increased to 112.5 μmol/L, then to 312.5 μmol/L, then 712.5 μmol/L, and finally to 1.4 mmol/L. The cell suspension was then centrifuged at 1,000×g for 3 min at room temperature. Cells were then inspected and counted, and if <60% of myocytes were rod-shaped (viable) the cells were not used for experimentation. Cardiomyocytes were then resuspended in plating medium [perfusion buffer with 2.5% (vol/vol) FBS, 1% (vol/vol) penicillin/streptomycin (P/S), and 1.4 mM CaCl₂, and plated on either 6 well plates (for myofibril mechanics) or 24 well plates (for hypertrophy imaging) coated with 9-10 μg/mL Laminin. Cells were incubated in plating buffer for 1-2 hours at room temperature to promote attachment; myocytes then were washed 2× in sterile PBS to remove dead cells, and media was changed to myocyte culture medium [MEM with 0.2% (wt/vol) BSA, 10 mM Hepes, 4 mM NaHCO₃ 10 mM creatine, 0.5% (vol/vol) insulinselenium-transferrin (Thermo Fisher Scientific, Waltham, Mass.), 1% (vol/vol) penicillin/streptomycin, 1 μM Blebbistatin (Cayman Chemicals, Ann Arbor, Mich.), filtered at 0.22 μm pH brought to 7.4, for 30-60 minutes before treatment.

Myofibril mechanics: Myofibril mechanics were performed on myofibrils isolated from human adult cardiac tissue, mouse ventricular cardiomyocytes, and stem cell derived cardiomyocytes. To isolate myofibrils from adult cardiac tissue, frozen heart sections were cut into ˜5 mm strips and skinned overnight in 0.5% Triton-X in rigor solution (132 mM NaCl, 5 mM KCl, 1 mM MgCl₂, 10 mM Tris, 5 mM EGTA, pH 7.1) containing protease inhibitors (10 μM leupeptin, 5 μM pepstatin, 200 μM PMSF and 10 μM E64), as well as 500 μM NaN₃ and 500 μM DTT at 4° C. These skinned LV strips were then washed several times in relaxing solution and homogenized (Tissue-Tearor, Thomas Scientific, Swedesboro, N.J.) in relaxing solution (pCa 9.0) containing protease inhibitors.

To isolate myofibrils from adult mouse cardiomyocytes and stem cell derived cardiomyocytes, cells were lysed and myofibrils prepared using an adaption from a recently described method. In brief, cells were lysed in relaxing solution with protease inhibitors and 20% (w/vol) sucrose, using a scraper. The lysed cell solution was then transferred into a microfuge tube and incubated on ice for 10 minutes with periodic vortexing. The cell/myofibril solution was then centrifuged at 1,500 g for 5 minutes at 4° C., and the cell/myofibril pellet resuspended in fresh relaxing solution with protease inhibitors. This process was repeated twice to remove any residual sucrose, and the cell/myofibril solution was then homogenized. A significantly more strenuous homogenization protocol was required for AMVCMs compared to hiPSC-CMs.

Myofibril mechanics were quantified using the fast solution switching technique on a custom-built experimental rig. Myofibril suspensions were plated onto a temperature controlled glass coverslip (15° C.) containing relaxing solution. Single myofibrils and myofibril bundles were then picked up from the surface of this coverslip and mounted between two glass micro-tools. One tool, the stretcher, was linked to a motor which could produce rapid changes in length (Mad City Labs, Madison, Wis.), while the second tool was a calibrated cantilevered force probe (6-8 μm/μN; frequency response 2-5 KHz), which was colored with black ink to increase probe sensitivity. After mounting myofibrils between microtools, they were stretched to 5-10% above slack myofibril length. Average sarcomere lengths and myofibril diameters were measured using ImageJ software. Mounted myofibrils were then activated and relaxed by rapidly changing the position of a double-barreled pipette simultaneously releasing activating solution (pCa 4.5) and relaxing solution (pCa 9.0) from each barrel. By rapidly changing the position of the pipette, the mounted myofibril could be induced to activate and relax. From the activation/relaxation trace produced, the following parameters were acquired: resting tension, the passive tension produced in relaxing conditions, induced by a push on the stretcher; maximum tension (mN/mm2), the greatest level of active tension generated by the myofibril at full calcium activation (pCa 4.5); activation kinetics (kAct), the rate constant of tension development following myofibril activation; reactivation kinetics (kTr), the rate constant of tension redevelopment following myofibril release/stretch to break cross bridges; slow phase relaxation kinetics (kREL,SLOVV), the kinetics of the initial slow/linear phase of myofibril relaxation, slow phase relaxation time (TLIN), the duration of the slow phase of relaxation, from the switching of the pipette to relaxing solution to the beginning of the fast phase of relaxation; fast phase relaxation kinetics (kREL,FAST), the rate constant of the fast (exponential) phase of relaxation.

Statistical Analysis: All data was first tested for normality. Data which did not initially demonstrate normality was then log2 transformed. Metabolomics data was log10 transformed instead, because log2 transformation did not achieve normality. For data which demonstrated normality (either before or after transformation), for two-group comparisons, an unpaired t-test was used. For multi-group comparisons, a one-way ANOVA with Tukey's multiple comparison test was used. For data which did not demonstrate normality after log transformation, for two group comparisons, a Mann-Whitney test was used. For multi-group comparisons, a Kruskal-Wallis test followed by Dunn's multiple comparison test was used.

P values or adjusted P values of less than 0.05 were considered to be significant. All data is displayed as mean plus or minus standard error the mean (SEM).

Venn's Diagrams were created using Venny 2.1. Heatmaps were created Heatmapper; samples were clustered by centroid linkage. All other statistical analysis was performed using GraphPad Prism 8.3.0 (GraphPad Software, San Diego, Calif.).

### Example 8—Discussion

The described metabolic screening data indicated that the most altered metabolites between more mature and immature cells were intermediates and products of glycolysis (higher in less mature cells), oleic and myristoleic acid, and intermediates of long chain fatty acid oxidation (higher in more mature cells) (FIG. 11). While it has been reported that oleic acid treatment alone induced a limited degree of metabolic maturity (in mouse cardiac precursor cells), it was surprising that the presently disclosed fatty acid media combined with patterned growth surfaces produced significantly more, and, even more surprisingly, induced an enhanced level of maturity. This may indicate that a shift from glycolytic energy production to a reliance on fatty acids, oleic and/or palmitic acid, is a beneficial part of this process. Changes in cardiolipin content were also observed with fatty acid supplementation and patterning, but whether this is a cause or consequence of maturity methods remains unknown.

While some have reported effects on maturation from growth on micro and nanopatterned substrates for hiPSC-CMs, the mechanism by which they do so remains significantly uncharacterized, the present results are surprising in the ability of the presently disclosed media, in combination with the larger and less regular patterning, to induce the type and magnitude of maturity. The presently disclosed results demonstrate that the disclosed patterned surfaces lead to alterations in cellular morphology (FIG. 8C), sarcomere organization (FIG. 9), and myofibril behavior (FIG. 10), in combination with change is the degree of improved metabolic maturity in MPAT cells vs MM. These surprising improvements were seen in cardiolipin remodeling (FIG. 11, 19) and fatty acid oxidization gene expression (FIG. 11A), suggesting, without wishing to be limited by theory, that growth on patterned surfaces has a beneficial effect on maturity, in terms of metabolic function.

Immature hiPSC-CMs and fetal cardiomyocytes typically have an atrial cell-like identity, with dominant expression of the atrial isoforms of myosin light and heavy chains, MLC-2A and MYH6, respectively. By contrast, expression of MYH7 was predominant in all hiPSC-CM cultures, and increased relative expression of MLC-2V to MLC-2A in MPAT hiPSC-CMs was also observed. The activation kinetics measured in all hiPSC-CMs conditions (0.95-1.04) are also lower than previously reported values in human atrial tissue (3.73) or in decellularized eSC-CM myofibrils expressing either mostly MYH7 or mixed MYH6/7 (1.70 or 2.44 respectively). Together, these findings indicate that the presently disclosed cells possess enhanced sarcomeric maturity and potentially a more ventricular-like identity. As forced expression of MLC-2V results in increased contractility in isolated cardiomyocytes, this could be, without wishing to be limited by theory, a mechanism of the increased myofibril force generation observed in the disclosed MPAT cells.

Previous reports have indicated that prolonged (>100 days) culture of hiPSC-CMs is sufficient to induce a more adult-like phenotype, as indicated by improvements in sarcomeric ultrastructure, cell morphology, and contractility. hiPSC-CMs cultured in glucose-containing media had no hypertrophic response to PE, high levels of BNP expression, and modest induction of hypertrophy in Danon hiPSC-CMs (FIG. 12, 20). The antihypertrophic agent JQ-1 also reduced cell area significantly beyond baseline in GLUC hiPSC-CMs, which did not occur in MM or MPAT cells (FIG. 12A-B). A recent report indicated that 14 days of culture in 22 mM glucose was sufficient to induce cardiac hypertrophy and dysfunction in hiPSC-CMs. Although the disclosed GLUC medium (RPMI-1640+B27Tm supplement) contains only 10.9 mM glucose, prolonged culture under these conditions may be sufficient to induce a hypertrophic phenotype. Similarly, it has been reported that supplementation of culture medium with either 5 or 20% fetal calf serum for one week completely masked the prohypertrophic effects of PE treatment in hiPSC-CMs 30-40 days post induction. Myofibrils isolated from GLUC cells also demonstrated low levels of active tension generation. Without wishing to be limited by theory, these findings could indicate lack of maturity of these cells, but could also potentially be a product of their hypertrophic phenotype as well: certain forms of cardiac dysfunction, such as Danon disease, are associated with reduced active tension generation.

hiPSC-CMs are increasingly used to model various types of cardiomyopathies, and these studies, along with the presently disclosed findings, indicate that a careful choice of media and culture conditions for long term culture is beneficial for in vitro characterization of hypertrophic cardiomyopathy.

While similar changes were observed in relaxation in PE-treated hiPSC-CMs and human HCM hearts, PE treatment did not dramatically induce an increase of activation kinetics. Without wishing to be limited by theory, this could be due to differences between the myofibrils from human heart and hiPSC-CMs: hiPSC-CMs show somewhat higher baseline activation kinetics (Table 1 and 5 versus 4). However, short term (48 hours) treatment with PE is also unlikely to fully recapitulate the changes which occur in human HCM, which may occur over many years. A recent report demonstrated that 48 hours of PE treatment on adult rat cardiac (ARVM) myofibrils had no effect on the kinetics of myofibril activation or relaxation, but caused increased myofibril calcium sensitivity. In contrast, disclosed methods providing 72 hours of PE treatment on AMVCMs resulted in a slight reduction in linear phase relaxation time (FIG. 21). Notably, the kinetics of human and rodent myofibrils are dissimilar: in rodent, activation and relaxation constants are much larger, and linear phase relaxation time is much shorter (˜50 msec in rodents versus 150-200 msec in human). The capability of PE to further shorten this already short linear phase, or to further increase activation or relaxation kinetics, may therefore be abrogated, leading to modest effects in rodent. PE treatment had a much greater effects on hiPSC-CMs, where linear phase was shortened by more than 80 msec, than AMVCMs, where it was only shortened by ˜10 msec (See Table 5 below versus Table 6).

TABLE 5
PE-treated hiPSC-CM Myofibril Characteristics

| | hiPSC-CM+Veh | hiPSC-CM+PE |
|---|---|---|
| k_{REL, SLOW} (s⁻¹) | 0.38 ± 0.07 | 0.58 ± 0.12 |
| t_{LIN} (mSec) | 196.0 ± 17.4 | 113.6 ± 6.2**** |
| k_{REL, FAST} (s⁻¹) | 6.23 ± 0.63 | 8.64 ± 0.77* |
| k_{ACT} (s⁻¹) | 0.88 ± 0.09 | 1.03 ± 0.08 |
| k_{TR} (s⁻¹) | 0.76 ± 0.04 | 0.80 ± 0.06 |
| Max. tens. (mN/mm²) | 47.96 ± 5.91 | 44.04 ± 5.05 |
| Rest. tens. (mN/mm²) | 7.06 ± 1.16 | 6.15 ± 0.90 |
| Diameter (μm) | 4.49 ± 0.26 | 4.48 ± 0.24 |
| N (# myofibrils) | 45 | 44 |

TABLE 6
PE-treated AMVCM Myofibril Characteristics

| | AMVCM+PBS | AMVCM+PE |
|---|---|---|
| k_{REL, SLOW} (s⁻¹) | 1.78 ± 0.62 | 1.83 ± 0.32 |
| t_{LIN} (mSec) | 57.15 ± 5.36 | 45.83 ± 2.58* |
| k_{REL, FAST} (s⁻¹) | 29.79 ± 3.46 | 36.61 ± 4.59 |
| k_{ACT} (s⁻¹) | 4.42 ± 0.35 | 4.45 ± 0.33 |
| k_{TR} (s⁻¹) | 3.57 ± 0.30 | 3.12 ± 0.30 |
| Max. tens. (mN/mm²) | 57.52 ± 5.04 | 68.55 ± 6.39 |
| Rest. tens. (mN/mm²) | 4.72 ± 0.71 | 6.94 ± 1.07 |
| Diameter (μm) | 4.66 ± 0.29 | 4.12 ± 0.22 |
| N (# myofibrils) | 21 | 26 |

The presently disclosed mature hiPSC-CMs may be an appropriate in vitro model to conduct translational research of human myofibril mechanics in response to pathological stresses. The systems and methods disclosed herein could also be of particular use when combined with CRISPR-based mutation strategies to study hypertrophic cardiomyopathy or other types of cardiomyopathies, allowing simultaneous assessment of cellular morphology, myofibril mechanics, metabolic behavior, and hypertrophic remodeling.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

We claim:

1. The method of claim 10, wherein the cardiomyocytes mimic adult human cardiomyocytes.

2. The method of claim 10, wherein the grooves are created using lapping paper.

3. The method of claim 10, wherein the lapping paper is 20 micron lapping paper.

4. The method of claim 10, wherein to improve culture surface sterility the surfaces are incubated for several hours in 100% ethanol, then under UV light.

5. The method of claim 10, wherein the culture grooves coated with fibronectin in order to improve cell attachment.

6. A population of in-vitro human induced pluripotent stem cell cardiomyocytes with enhanced maturity, comprising;
sarcomeres with an average length of greater than about 1.9 μm, and myofibrils that exert a maximum tension of greater than about 40 mN/mm².

7. The population of in-vitro induced human pluripotent stem cell cardiomyocytes of claim 6, wherein the cells are grown in media comprising at least one of linoleic, palmitic, or oleic acid and on a growth surface having grooves of at least about 1 μm.

8. The population of in-vitro human induced pluripotent stem cell cardiomyocytes of claim 7, wherein the cells are elongated relative to cells grown on non-grooved growth surfaces.

9. The population of in-vitro human induced pluripotent stem cell cardiomyocytes of claim 7, wherein the cells are less circular than cells grown on non-grooved growth surfaces.

10. A method of producing a human induced pluripotent stem cell cardiomyocytes with enhanced maturity comprising:
inducing a human pluripotent stem cell to differentiate into a plurality of cardiomyocytes;
culturing the cardiomyocytes in a medium comprising galactose and one or more of linoleic, palmitic and oleic acid;
culturing the cardiomyocytes on a growth surface comprising a pattern having grooves of about 1-40 microns, wherein the cardiomyocytes produce sarcomeres with enhanced structure and function.

11. The method of claim 10, wherein the cells comprise sarcomeres with an average length of greater than about 1.9 μm, and myofibrils that exert a maximum tension of greater than about 40 mN/mm².

12. The method of claim 10, wherein the concentration of one of linoleic, palmitic, or oleic acid in the media is between about 10 μM and 200 μM.

13. The method of claim 10, wherein the concentration of galactose is 10 mM.

14. The method of claim 10, wherein the grooves are about 20 μm.

15. The method of claim 10, wherein the cells are elongated relative to cells grown on non-grooved growth surfaces.

16. The method of claim 10, wherein the cells are less circular than cells grown on non-grooved growth surfaces.

17. A method of using a human induced pluripotent stem cell cardiomyocytes with enhanced maturity comprising:
culturing the cardiomyocytes in a medium comprising galactose and one or more of linoleic, palmitic and oleic acid;
culturing the cardiomyocytes on a growth surface comprising a pattern having grooves of about 1-40 microns; and
allowing the cardiomyocytes to form myofibrils;
administering a pharmaceutical compound to the medium; and
comparing at least one characteristic of the cardiomyocyte to a human induced pluripotent stem cell cardiomyocyte grown under similar conditions in the absence of the pharmaceutical compound.

18. The method of claim 17, wherein the characteristic is selected from maximum myofibril tension, resting myofibril tension, myofibril contraction kinetics, circularity, sarcomere length, gene expression, or protein expression.

19. The method of claim 17, wherein the characteristic is maximum myofibril tension.

20. The method of claim 17, wherein the human induced pluripotent stem cell cardiomyocytes is obtained from a subject having or at risk of developing heart disease.
